(19) United States
(12) Patent Application Publication
(10) Pub. No.: US 2023/0374606 A1
(43) Pub. Date: Nov. 23, 2023

(54) METHODS FOR TREATING MELANOMA

(71) Applicant: UNIVERSITY OF LOUISVILLE RESEARCH FOUNDATION, INC., Louisville, KY (US)

(72) Inventors: Kelly M. MCMASTERS, Crestwood, KY (US); Hongying HAO, Marysville, IN (US); Austin J. MCMASTERS, Louisville, KY (US)

(73) Assignee: UNIVERSITY OF LOUISVILLE RESEARCH FOUNDATION, INC., Louisville, KY (US)

(21) Appl. No.: 18/246,702

(22) PCT Filed: Oct. 1, 2021
(86) PCT No.: PCT/US2021/071673

§ 371 Date: Mar. 27, 2023

Related U.S. Application Data
(60) Provisional application No. 63/086,613, filed on Oct. 2, 2020.

(51) Int. Cl. C12Q 1/6886; C12Q 1/6851

(52) U.S. Cl. CPC C12Q 1/6886; C12Q 1/6851; C12Q 2600/158; C12Q 2600/118

(57) ABSTRACT

Some embodiments of the invention include methods for treating melanoma in a subject. In other embodiments, the methods for treating melanoma in a subject comprise quantifying an RNA expression level for at least one biomarker in a sample (e.g., from a sentinel lymph node of the subject) and administering to the subject a treatment for melanoma (e.g., by administering to the subject immunotherapy, interferon, a BRAF inhibitor, a checkpoint inhibitor, a Wnt10b inhibitor, an IRAK3 inhibitor, or a combination thereof). In still other embodiments, the at least one biomarker can comprise FOS, NR4A, ITGB1, IRAK3, Wnt10b, or a combination thereof. Additional embodiments of the invention are also discussed herein.

### CROSS-REFERENCE TO RELATED APPLICATIONS

This application claims the benefit of U.S. Provisional Application No. 63/086,613, filed Oct. 2, 2020, entitled “Methods for Treating Diseases” which is herein incorporated by reference in its entirety.

### BACKGROUND

Melanoma is less common than some other skin cancers, but it is one of the more dangerous forms of skin cancer. To date, treatment of melanoma is limited. Accordingly, some embodiments of the present invention include treating melanoma. Additional embodiments of the invention are also discussed herein.

### SUMMARY

Some embodiments of the present invention include methods for treating melanoma in a subject, the method comprising quantifying an RNA expression level for at least one biomarker in a sample from a sentinel lymph node of the subject, where the at least one biomarker comprises FOS, NR4A, ITGB1, IRAK3, Wnt10b, or a combination thereof, and administering to the subject immunotherapy, interferon, a BRAF inhibitor, a checkpoint inhibitor, a Wnt10b inhibitor, an IRAK3 inhibitor, or a combination thereof.

In some embodiments, the at least one biomarker comprises NR4A1, NR4A2, NR4A3, or a combination thereof. In certain embodiments, the at least one biomarker comprises NR4A2, NR4A3, or both. In other embodiments, the at least one biomarker comprises NR4A, FOS, Wn10b, or a combination thereof. In still other embodiments, the at least one biomarker comprises NR4A. In yet other embodiments, the at least one biomarker comprises IRAK3. In certain embodiments, the at least one biomarker further comprises SFRP2, SFRP4, PTGS2, LINC00518, IL1B, or a combination thereof. In some embodiments, the at least one biomarker further comprises a biomarker listed in Table 2, Table 3, Table 4, Table 5, Table 6, Table 7, Table 8, Table 9, Table 10, Supp. Table 2, Supp. Table 3, Supp. Table 4, Supp. Table 5, Supp. Table 6, or a combination thereof. In other embodiments, the at least one biomarker further comprises ACVR1C, ACVR2A, ALCAM, ALKBH2, ATG7, ATP2B2, BACH2, BAGE, BCOR, BID, C1QBP, C3, C6, CASP9, CCL16, CCL18, CCL4, CD244, CD84, CD8A, CDK4, CDK6, CDKN1A, CLEC4C, CLEC7A, COL28A1, COMP, CTNNB1, CXL/CXCR, CXCL3, CXCL5, CXCR4, CYBB, DKK2, DLK1, DLL4, DNMT3A, DOCK9, DUSP1, ELK1, EPOR, ERCC2, ERCC6, ERGIC3, F2RL1, FANCB, FANCL, FOS, FOSB, FUT8, GADD45A, GRB14, GRIK2, HHEX, HLA-A, HLA-DMA, HLA-DMB, HLA-G, HSP90B1, HSPA1A, ICAM1, IDO1, IFIH1, IFITM1, IGF1R, IL1B, IL23R, IL6, INHBA, INPP5D, IRAK3, ITGB1, ITGBL1, JAM3, KLF4, KLRC4-KLRK1///KLRK1, LAMA5, LIF, LINC00354, LINC00518, LIX1, LOC100507516, LOC101928963, LOC105373225, MAGEA3, MAP2K2, MAP2K4, MAPK11, MAVS, MIF, MKX, MLANA, MME, MS4A6A, MST1R, MUC15, MX1, NCAM1, NFKBIZ, NKD1, NOD1, NOG, NR4A, NR4A1, NR4A2, NR4A3, NRCAM, PBRM1, PCNA, PIK3CB, PIK3R3, PLAU, PLD1, PPP2R1A, PRKAR2A, PRUNE2, PSMB8, PTGS2, RAD50, RB1, RELA, RGS1, RNF152, RPS6KA5, RUNX1, S100B, SATB1, SFRP2, SFRP4, SLC13A5, SMAD2, SOS1, SPINK5, STK11, TBK1, TFDP1, TFPI2, TGFB3, TLR10, TLR6, TNC, TNFRSF10C, TNFRSF13B, UBB, WNT10B, WWC1, XRCC4, or a combination thereof.

In some embodiments, the quantifying is carried out using polymerase chain reaction, real-time polymerase chain reaction, reverse transcriptase polymerase chain reaction, real-time quantitative RT-PCR, microarray, NanoString, or a combination thereof. In other embodiments, the quantifying is carried out using polymerase chain reaction, real-time polymerase chain reaction, reverse transcriptase polymerase chain reaction, real-time quantitative RT-PCR, or a combination thereof.

In certain embodiments, the subject is no more than about 30 years old, no more than about 40 years old, no more than about 50 years old, no more than about 60 years old, or no more than about 70 years old. In other embodiments, the subject is at least about 40 years old, at least about 50 years old, at least about 60 years old, at least about 70 years old, or at least about 80 years old.

In some embodiments, the subject has a positive sentinel lymph node status.

In other embodiments, the quantifying is relative to a control sample where the melanoma was without recurrence for at least about 5.0 years.

In yet other embodiments, the fold change in the RNA expression level, relative to a control sample where the melanoma was without recurrence for at least about 5.0 years, in one or more of the at least one biomarker is at least about 1.0, at least about 1.5, at least about 2.0, at least about 2.5, at least about 3.0, at least about 3.5, at least about 4.0, or at least about 4.5.

In some embodiments, the method further comprises assessing a clinicopathologic feature of the subject from which the sample was obtained. In certain embodiments, the method further comprises assessing a clinicopathologic feature of the subject from which the sample was obtained and the clinicopathologic feature is age, gender, anatomic location, Breslow thickness, ulceration, sentinel lymph node status, or a combination thereof. In other embodiments, the method further comprises assessing a clinicopathologic feature of the subject from which the sample was obtained and the clinicopathologic feature is metastasis, age, lesion site, tumor burden, number of positive nodes, ulceration, tumor thickness, or a combination thereof.

In some embodiments, the subject has stage III melanoma.

In certain embodiments, the administering comprises administering interferon, interferon alfa-2b, or both. In other embodiments, the administering comprises administering a BRAF inhibitor, vemurafenib, dabrafenib, trametinib, encorafenib, or a combination thereof. In still other embodiments, the administering comprises administering a checkpoint inhibitor, a PD-1 inhibitor, nivolumab, pembrolizumab, cemiplimab, a PD-L1 inhibitor, atezolizumab, avelumab, durvalumab, a cytotoxic T-lymphocyte antigen 4 inhibitor, ipilimumab, or a combination thereof. In yet other embodiments, the administering comprises administering a Wnt10b inhibitor, DKN-01, CGX1321, ETC 1922159, or a combination thereof. In certain embodiments, (a) the administering comprises administering a Wnt10b inhibitor, DKN-01, CGX1321, ETC 1922159 or a combination thereof and (b) the subject is at least about 60 years old. In some embodiments, the administering comprises administering an IRAK3 inhibitor, pacritinib, thymoquinone, or a combination thereof. In other embodiments, (a) the administering comprises administering an IRAK3 inhibitor, pacritinib, thymoquinone, or a combination thereof and (b) the subject is no more than about 60 years old.

In certain embodiments, the administering only occurs if the fold change in the RNA expression level, relative to a control sample where the melanoma was without recurrence for at least about 5.0 years, in one or more of the at least one biomarker is at least about 1.0, at least about 1.5, at least about 2.0, at least about 2.5, at least about 3.0, at least about 3.5, at least about 4.0, or at least about 4.5.

In yet other embodiments, the administering comprises administering a Wnt10b inhibitor, DKN-01, CGX1321, ETC 1922159, or a combination thereof, and the administering only occurs if (a) the fold change in the RNA expression level, relative to a control sample where the melanoma was without recurrence for at least about 5.0 years, in one or more of the at least one biomarker is at least about 1.0, at least about 1.5, at least about 2.0, at least about 2.5, at least about 3.0, at least about 3.5, at least about 4.0, or at least about 4.5 and (b) the subject is at least about 60 years old.

In some embodiments, the administering comprises administering of IRAK3 inhibitor, pacritinib, thymoquinone, or a combination thereof, and the administering only occurs if (a) the fold change in the RNA expression level, relative to a control sample where the melanoma was without recurrence for at least about 5.0 years, in one or more of the at least one biomarker is at least about 1.0, at least about 1.5, at least about 2.0, at least about 2.5, at least about 3.0, at least about 3.5, at least about 4.0, or at least about 4.5 and (b) the subject is no more than about 60 years old.

In still other embodiments, the treating further comprises one or more of surgery, chemotherapy, radiation therapy, targeted therapy, or vaccine therapy.

In certain embodiments, the subject is a mammal, a primate, or a human. In other embodiments, the subject is a human.

In other embodiments, the method comprises quantifying an RNA expression level for at least one biomarker in a sample from a sentinel lymph node of the human, where the at least one biomarker comprises FOS, NR4A, Wnt10b, or a combination thereof, and administering to the human a Wnt10b inhibitor, DKN-01, CGX1321, ETC 1922159, or a combination thereof, and optionally one or more of immunotherapy, interferon, a BRAF inhibitor, a checkpoint inhibitor, or an IRAK3 inhibitor; the human is at least about 60 years old, and the administering only occurs if the fold change in the RNA expression level, relative to a control sample where the melanoma was without recurrence for at least about 5.0 years, in one or more of the at least one biomarker is at least about 1.5.

In still other embodiments, the method comprises quantifying an RNA expression level for at least one biomarker in a sample from a sentinel lymph node of the human, where the at least one biomarker comprises IRAK3 and administering to the human an IRAK3 inhibitor, pacritinib, thymoquinone, or a combination thereof, and optionally one or more of immunotherapy, interferon, a BRAF inhibitor, a checkpoint inhibitor, or a Wnt10b inhibitor; the human is no more than about 60 years old, and the administering only occurs if the fold change in the RNA expression level, relative to a control sample where the melanoma was without recurrence for at least about 5.0 years, in one or more of the at least one biomarker is at least about 1.5.

Other embodiments of the invention are also discussed herein.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1: The network connection of the 156 DEGs in the older versus the younger patients by microarray T4 filter.

FIG. 2: A schematic model showing that the DEGs in the older melanoma patients by recurrence status converge at the Wnt signaling pathway.

### DETAILED DESCRIPTION

Some embodiments of the invention include methods for treating melanoma in a subject (e.g., primate or human), the method comprising quantifying an RNA expression level for at least one biomarker in a sample from a sentinel lymph node (SLN) of the subject and then treating the subject (e.g., by administering one or more molecules to the subject). In certain embodiments, the at least one biomarker comprises one or more of FOS, NR4A, ITGB1, IRAK3, or Wnt10b. In other embodiments, treating the subject comprises administering to the subject with one or more of immunotherapy, interferon, a BRAF inhibitor, a checkpoint inhibitor, a Wnt10b inhibitor, or an IRAK3 inhibitor.

Any suitable method for RNA extraction can be used. In some embodiments, RNA isolation can be performed using purification kit, buffer set and protease from commercial manufacturers according to the manufacturer's instructions. In other embodiments, total RNA from cells in culture can be isolated using Qiagen RNeasy mini-columns. Numerous RNA isolation kits are commercially available and can be used in the methods of the invention.

The method for quantifying an RNA expression level can be any suitable method including but not limited to using a microarray, NanoString (e.g., NanoString technologies, Seattle, WA, USA), real-time polymerase chain reaction (RT-PCR), real time quantitative PCR (e.g., which measures PCR product accumulation through a dual-labeled fluorogenic probe), quantitative competitive PCR (e.g., where internal competitor for each target sequence is used for normalization), quantitative comparative PCR (e.g., which uses a normalization gene contained within the sample), or a combination thereof. In certain embodiments, the quantifying is carried out using polymerase chain reaction, real-time polymerase chain reaction, reverse transcriptase polymerase chain reaction, real-time quantitative RT-PCR, microarray, NanoString, or a combination thereof. In other embodiments, the quantifying is carried out using polymerase chain reaction, real-time polymerase chain reaction, reverse transcriptase polymerase chain reaction, real-time quantitative RT-PCR, or a combination thereof.

In some instances, one of the first steps in gene expression profiling by RT-PCR is the reverse transcription of the RNA template into cDNA, followed by amplification in a PCR reaction. Reverse transcriptases include, but are not limited to, avilo myeloblastosis virus reverse transcriptase (AMV-RT) and Moloney murine leukemia virus reverse transcriptase (MMLV-RT). The reverse transcription step is sometimes primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it sometimes employs the Taq DNA polymerase, which has a 5′-3′ nuclease activity but lacks a 3′-5′ proofreading endonuclease activity. TaqMan PCR sometimes utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan™ RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany) In one specific embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is sometimes performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH), Beta-2-microglobulin (B2M), and (3-actin.

Another variation of the RT-PCR technique is the real time quantitative PCR, which measures PCR product accumulation through a dual-labeled fluorogenic probe (e.g., TaqMan™ probe). Real time PCR is compatible both with quantitative competitive PCR, where internal competitor for each target sequence is used for normalization, and with quantitative comparative PCR using a normalization gene contained within the sample, or a housekeeping gene for RT-PCR.

When conducting RT-PCR analysis, an initial step is the isolation of mRNA from the sample. The starting material is sometimes total RNA isolated from a SLN. mRNA can be extracted, for example, from frozen or archived paraffin-embedded and fixed (e.g., formalin-fixed) tissue samples.

The route of administration for any of the treatments of the invention can be of any suitable route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, subcutaneous administration, topical administration, intradermal administration, oral administration, sublingual administration, intranasal administration, or intramuscular administration. The choice of administration route can depend on the molecule used for treatment (e.g., inhibitor), the physical and chemical properties of the molecule used for treatment, as well as the age and weight of the subject (e.g., human), the particular melanoma, the severity of the melanoma, and the stage of the melanoma. Of course, combinations of administration routes can be administered, as desired.

In some embodiments, melanoma is a tumor arising from the melanocytic system of the skin and other organs. In other embodiments, melanomas can include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungual melanoma, and superficial spreading melanoma.

In some embodiments, the sample obtained from a sentinel lymph node refers to a sample that comprises a biomolecule and/or is derived from a sentinel lymph node of the animal. In certain embodiments, biomolecules can include, but are not limited to total DNA, RNA, miRNA, mRNA, and polypeptides. The sample can be used for the detection of the presence and/or expression level of a biomolecule of interest (e.g., biomarker). Any suitable portion of the lymph node can be used with the methods (e.g., described herein), such as but not limited to biopsy, tissue, tissue section, cell, group of cells, cell fragment, or cell product from the lymph node. In some embodiments, the sample can be provided as a frozen or fresh cell or tissue sample (e.g., paraffin-embedded tissue). In some embodiments, the sample can be provided as an extract (e.g., mRNA extracted from cell or tissue).

In certain embodiments, the sample obtained from the SLN, or the SLN from which the sample is obtained, can be acquired at a time when sentinel nodes would be normally identified and removed, for example at or around the time of surgery to remove a primary melanoma. In some embodiments, it can be desirable to use a fresh sample, or a paraffin-embedded tissue sample. In other embodiments, it can be desirable to freeze or otherwise store for use at a later date. In still other embodiments, it can be useful to process (e.g., extract) the sample, using a portion for immediate testing and/or saving a portion for use at a later date.

In some embodiments, the subject is an animal or is a vertebrate animal, such as but not limited to a warm-blooded vertebrate, a mammal, primate or a human. In certain embodiments, veterinary therapeutic uses are provided in accordance with the presently disclosed subject matter. As such, the presently disclosed subject matter provides methods related to mammals such as humans, as well as those mammals (e.g., primates) of importance due to being endangered, such as Siberian tigers; of economic importance, such as animals raised by humans; and/or animals of social importance to humans, such as animals kept as pets or in zoos. Examples of such animals include but are not limited to: carnivores (e.g., cats and dogs); swine (e.g., pigs, hogs, and wild boars); ruminants and/or ungulates (e.g., cattle, oxen, sheep, giraffes, deer, goats, bison, and camels); and horses (e.g., race horses). In some embodiments, the animal is a human.

In some embodiments, the subject has a single positive sentinel lymph node. In some embodiments, the subject is classified or diagnosed with stage III melanoma; classification with stage III melanoma can occur when there is a presence of at least one positive sentinel lymph node.

In some embodiments, the at least one biomarker for which RNA expression level is quantified can be any suitable biomarker or set of biomarkers. In certain embodiments, the number of biomarkers quantified can be 1, 2, 3, 4, 5, 6, 7, 8, 9, 10, 11, 12, 13, 14, 15, 16, 17, 18, 19, 20, 21, 22, 23, 24, 25, 26, 27, 27, 29, or 30. In some embodiments, the at least one biomarker comprises one or more of FOS, NR4A, ITGB1, IRAK3, or Wnt10b. In other embodiments, the at least one biomarker comprises two or more of FOS (FBJ murine osteosarcoma viral oncogene homolog), NR4A (nuclear receptor subfamily 4, group A), or ITGB1 (Integrin subunit beta 1). In yet other embodiments, the at least one biomarker comprises NR4A1, NR4A2, NR4A3, or a combination thereof. In certain embodiments, the at least one biomarker comprises NR4A2, NR4A3, or both. In other embodiments, the at least one biomarker comprises NR4A, FOS, Wn10b, or a combination thereof. In some embodiments, the at least one biomarker comprises NR4A. In other embodiments, the at least one biomarker comprises IRAK3. In still other embodiments, the at least one biomarker further comprises SFRP2, SFRP4, PTGS2, LINC00518, IL1B, or a combination thereof. In yet other embodiments, the at least one biomarker further comprises a biomarker listed in Table 2, Table 3, Table 4, Table 5, Table 6, Table 7, Table 8, Table 9, Table 10, Supp. Table 2, Supp. Table 3, Supp. Table 4, Supp. Table 5, Supp. Table 6, or a combination thereof. In some embodiments, the at least one biomarker further comprises ACVR1C, ACVR2A, ALCAM, ALKBH2, ATG7, ATP2B2, BACH2, BAGE, BCOR, BID, C1QBP, C3, C6, CASP9, CCL16, CCL18, CCL4, CD244, CD84, CD8A, CDK4, CDK6, CDKN1A, CLEC4C, CLEC7A, COL28A1, COMP, CTNNB1, CXL/CXCR, CXCL3, CXCL5, CXCR4, CYBB, DKK2, DLK1, DLL4, DNMT3A, DOCK9, DUSP1, ELK1, EPOR, ERCC2, ERCC6, ERGIC3, F2RL1, FANCB, FANCL, FOS, FOSB, FUT8, GADD45A, GRB14, GRIK2, HHEX, HLA-A, HLA-DMA, HLA-DMB, HLA-G, HSP90B1, HSPA1A, ICAM1, IDO1, IFIH1, IFITM1, IGF1R, IL1B, IL23R, IL6, INHBA, INPP5D, IRAK3, ITGB1, ITGBL1, JAM3, KLF4, KLRC4-KLRK1///KLRK1, LAMA5, LIF, LINC00354, LINC00518, LIX1, LOC100507516, LOC101928963, LOC105373225, MAGEA3, MAP2K2, MAP2K4, MAPK11, MAVS, MIF, MKX, MLANA, MME, MS4A6A, MST1R, MUC15, MX1, NCAM1, NFKBIZ, NKD1, NOD1, NOG, NR4A, NR4A1, NR4A2, NR4A3, NRCAM, PBRM1, PCNA, PIK3CB, PIK3R3, PLAU, PLD1, PPP2R1A, PRKAR2A, PRUNE2, PSMB8, PTGS2, RAD50, RB1, RELA, RGS1, RNF152, RPS6KA5, RUNX1, S100B, SATB1, SFRP2, SFRP4, SLC13A5, SMAD2, SOS1, SPINK5, STK11, TBK1, TFDP1, TFPI2, TGFB3, TLR10, TLR6, TNC, TNFRSF10C, TNFRSF13B, UBB, WNT10B, WWC1, XRCC4, or a combination thereof.

TABLE A
Abbreviations for Biomarkers

| | |
|---|---|
| ACVR1C | activin A receptor, type IC |
| ACVR2A | activin A receptor, type IIA |
| ALCAM | activated leukocyte cell adhesion molecule |
| ALKBH2 | alkB, alkylation repair homolog 2 (E. coli) |
| ATG7 | autophagy related 7 |
| ATP2B2 | ATPase, Ca++ transporting, plasma membrane 2 |
| BACH2 | BTB and CNC homology 1, basic leucine zipper transcription |
| | factor 2 |
| BAGE | B melanoma antigen |
| BCOR | BCL6 corepressor |
| BID | BH3 interacting domain death agonist |
| C1QBP | complement component 1, q subcomponent binding protein |
| C3 | complement component 3 |
| C6 | complement component 6 |
| CASP9 | caspase 9, apoptosis-related cysteine peptidase |
| CCL16 | chemokine (C-C motif) ligand 16 |
| CCL18 | chemokine (C-C motif) ligand 18 (pulmonary and activation- |
| | regulated) |
| CCL4 | chemokine (C-C motif) ligand 4 |
| CD244 | CD244 molecule, natural killer cell receptor 2B4 |
| CD84 | CD84 molecule |
| CD8A | CD8a molecule |
| CDK4 | cyclin-dependent kinase 4 |
| CDK6 | cyclin-dependent kinase 6 |
| CDKN1A | cyclin-dependent kinase inhibitor 1A (p21, Cip1) |
| CLEC4C | C-type lectin domain family 4, member C |
| CLEC7A | C-type lectin domain family 7, member A |
| COL28A1 | collagen, type XXVIII, alpha 1 |
| COMP | cartilage oligomeric matrix protein |
| CTNNB1 | catenin (cadherin-associated protein), beta 1, 88 kDa |
| CXL/CXCR | C-X-C Motif Chemokine Ligand |
| CXCL3 | chemokine (C-X-C motif) ligand 3 |
| CXCL5 | chemokine (C-X-C motif) ligand 5 |
| CXCR4 | chemokine (C-X-C motif) receptor 4 |
| CYBB | cytochrome b-245, beta polypeptide |
| DKK2 | dickkopf WNT signaling pathway inhibitor 2 |
| DLK1 | delta-like 1 homolog (Drosophila) |
| DLL4 | delta-like 4 (Drosophila) |
| DNMT3A | DNA (cytosine-5-)-methyltransferase 3 alpha |
| DOCK9 | dedicator of cytokinesis 9 |
| DUSP1 | dual specificity phosphatase 1 |
| ELK1 | ELK1, member of ETS oncogene family |
| EPOR | erythropoietin receptor |
| ERCC2 | excision repair cross-complementing rodent repair deficiency, |
| | complementation group 2 |
| ERCC6 | excision repair cross-complementing rodent repair deficiency, |
| | complementation group 6 |
| ERGIC3 | ERGIC and Golgi 3 |
| F2RL1 | coagulation factor II (thrombin) receptor-like 1 |
| FANCB | Fanconi anemia, complementation group B |
| FANCL | Fanconi anemia, complementation group L |
| FOS | FBJ murine osteosarcoma viral oncogene homolog |
| FOSB | FBJ murine osteosarcoma viral oncogene homolog B |
| FUT8 | fucosyltransferase 8 (alpha (1,6) fucosyltransferase) |
| GADD45A | growth arrest and DNA-damage-inducible, alpha |
| GRB14 | growth factor receptor bound protein 14 |
| GRIK2 | glutamate receptor, ionotropic, kainate 2 |
| HHEX | hematopoietically expressed homeobox |
| HLA-A | major histocompatibility complex, class I, A |
| HLA-DMA | major histocompatibility complex, class II, DM alpha |
| HLA-DMB | major histocompatibility complex, class II, DM beta |
| HLA-G | major histocompatibility complex, class I, G |
| HSP90B1 | heat shock protein 90 kDa beta (Grp94), member 1 |
| HSPA1A | heat shock 70 kDa protein 1A |
| ICAM1 | intercellular adhesion molecule 1 |
| IDO1 | indoleamine 2,3-dioxygenase 1 |
| IFIH1 | interferon induced with helicase C domain 1 |
| IFITM1 | interferon induced transmembrane protein 1 |
| IGF1R | insulin-like growth factor 1 receptor |
| IL1B | interleukin 1 beta |
| IL23R | interleukin 23 receptor |
| IL6 | interleukin 6 (interferon, beta 2) |
| INHBA | inhibin beta A |
| INPP5D | inositol polyphosphate-5-phosphatase, 145 kDa |
| IRAK3 | interleukin-1 receptor-associated kinase 3 |
| ITGB1 | integrin subunit beta 1 |
| ITGBL1 | integrin beta like 1 |
| JAM3 | junctional adhesion molecule 3 |
| KLF4 | Kruppel-like factor 4 (gut) |
| KLRC4- | KLRC4-KLRK1 read through /// killer cell lectin-like receptor |
| KLRK1///KLRK1 | subfamily K, member 1 |
| LAMA5 | laminin, alpha 5 |
| LIF | leukemia inhibitory factor |
| LINC00354 | long intergenic non-protein coding RNA 354 |
| LINC00518 | long intergenic non-protein coding RNA 518 |
| LIX1 | limb and CNS expressed 1 |
| LOC100507516 | uncharacterized LOC100507516 |
| LOC101928963 | uncharacterized LOC101928963 |
| LOC105373225 | uncharacterized LOC105373225 |
| MAGEA3 | melanoma antigen family A, 3 |
| MAP2K2 | mitogen-activated protein kinase kinase 2 |
| MAP2K4 | mitogen-activated protein kinase kinase 4 |
| MAPK11 | mitogen-activated protein kinase 11 |
| MAVS | mitochondrial antiviral signaling protein |
| MIF | macrophage migration inhibitory factor (glycosylation-inhibiting |
| | factor) |
| MKX | mohawk homeobox |
| MLANA | melan-A |
| MME | membrane metallo-endopeptidase |
| MS4A6A | membrane-spanning 4-domains, subfamily A, member 6A |
| MST1R | macrophage stimulating 1 receptor (c-met-related tyrosine |
| | kinase) |
| MUC15 | mucin 15, cell surface associated |
| MX1 | myxovirus (influenza virus) resistance 1, interferon-inducible |
| | protein p78 (mouse) |
| NCAM1 | neural cell adhesion molecule 1 |
| NFKBIZ | nuclear factor of kappa light polypeptide gene enhancer in B-cells |
| | inhibitor, zeta |
| NKD1 | naked cuticle homolog 1 (Drosophila) |
| NOD1 | nucleotide-binding oligomerization domain containing 1 |
| NOG | noggin |
| NR4A | nuclear receptor subfamily 4A |
| NR4A1 | nuclear receptor subfamily 4, group A, member 1 |
| NR4A2 | nuclear receptor subfamily 4, group A, member 2 |
| NR4A3 | nuclear receptor subfamily 4, group A, member 3 |
| NRCAM | neuronal cell adhesion molecule |
| PBRM1 | polybromo 1 |
| PCNA | proliferating cell nuclear antigen |
| PIK3CB | phosphatidylinositol-4,5-bisphosphate 3-kinase, catalytic subunit |
| | beta |
| PIK3R3 | phosphoinositide-3-kinase, regulatory subunit 3 (gamma) |
| PLAU | plasminogen activator, urokinase |
| PLD1 | phospholipase D1, phosphatidylcholine-specific |
| PPP2R1A | protein phosphatase 2, regulatory subunit A, alpha |
| PRKAR2A | protein kinase, cAMP-dependent, regulatory, type II, alpha |
| PRUNE2 | prune homolog 2 (Drosophila) |
| PSMB8 | proteasome (prosome, macropain) subunit, beta type, 8 (large |
| | multifunctional peptidase 7) |
| PTGS2 | Prostaglandin-Endoperoxide Synthase 2 (also named COX2) |
| RAD50 | RAD50 homolog (S. cerevisiae) |
| RB1 | retinoblastoma 1 |
| RELA | v-rel reticuloendotheliosis viral oncogene homolog A (avian) |
| RGS1 | Regulator of G-protein signaling 1 |
| RNF152 | ring finger protein 152 |
| RPS6KA5 | ribosomal protein S6 kinase, 90 kDa, polypeptide 5 |
| RUNX1 | runt-related transcription factor 1 |
| S100B | S100 calcium binding protein B |
| SATB1 | SATB homeobox 1 |
| SFRP2 | secreted frizzled-related protein 2 |
| SFRP4 | secreted frizzled-related protein 4 |
| SLC13A5 | solute carrier family 13 (sodium-dependent citrate transporter), |
| | member 5 |
| SMAD2 | SMAD family member 2 |
| SOS1 | son of sevenless homolog 1 (Drosophila) |
| SPINK5 | serine peptidase inhibitor, Kazal type 5 |
| STK11 | serine/threonine kinase 11 |
| TBK1 | TANK-binding kinase 1 |
| TFDP1 | transcription factor Dp-1 |
| TFPI2 | tissue factor pathway inhibitor 2 |
| TGFB3 | transforming growth factor, beta 3 |
| TLR10 | toll-like receptor 10 |
| TLR6 | toll-like receptor 6 |
| TNC | tenascin C |
| TNFRSF10C | tumor necrosis factor receptor superfamily, member 10c, decoy |
| | without an intracellular domain |
| TNFRSF13B | tumor necrosis factor receptor superfamily, member 13B |
| UBB | ubiquitin B |
| WNT10B | wingless-type MMTV integration site family, member 10B |
| WWC1 | WW and C2 domain containing 1 |
| XRCC4 | X-ray repair complementing defective repair in Chinese hamster |
| | cells 4 |

In some embodiments, the subject (e.g., human) is no more than about 30 years old, no more than about 40 years old, no more than about 50 years old, no more than about 60 years old, or no more than about 70 years old. In other embodiments, the subject (e.g., human) is at least about 40 years old, at least about 50 years old, at least about 60 years old, at least about 70 years old, or at least about 80 years old. In certain embodiments, the subject is about 10, about 15, about 20 , about 25, about 30, about 35, about 40, about 42, about 44, about 45, about 46, about 48, about 50, about 51, about 52, about 53, about 54, about 55, about 56, about 57, about 58, about 59, about 60, about 61, about 62, about 63, about 64, about 65, about 66, about 67, about 68, about 69, about 70, about 72, about 74, about 75, about 76, about 78, about 80, about 85, or about 90 years old.

In some embodiments, the subject (e.g., human) has a positive sentinel lymph node status.

The term “control sample” is used herein to refer to a reference to which a sample can be compared. In other embodiments, the control sample can be a reference standard. In certain embodiments, a reference standard can be a manufactured control sample, for example, designed to include a predetermined presence or amount of one or more biomarkers to which a sample can be compared. In some embodiments, a reference standard can comprise a compilation about the presence and/or level of one or more biomarkers considered to be control values. In some embodiments, the control sample can be a sample obtained from a sentinel lymph node of a control subject. A “control subject” can be selected with consideration to the subject being tested. In some embodiments, a control subject can be a subject in which melanoma has not recurred for a period of about 3.0, about 4.0, about 5.0, about 6.0, about 7.0, about 8.0, about 9.0, about 10.0, or more years. In some embodiments, a control subject can be a subject which has been non-symptomatic for a period of about 3.0, about 4.0, about 5.0, about 6.0, about 7.0, about 8.0, about 9.0, about 10.0, or more years. In some embodiments, a control subject can be a subject which is free of melanoma. In some embodiments, the control sample can be an average or composite value based on analysis of a population of “control subjects.”

In other embodiments, the quantifying is relative to a control sample where the melanoma was without recurrence for at least about 5.0 years. In some embodiments, the control sample is obtained from a control subject (e.g., a human or a primate), where the control subject is the same type of animal as the subject (e.g., human or primate).

In some embodiments, the fold change in the RNA expression level, relative to a control sample where the melanoma was without recurrence for at least about 5.0 years, in one or more of the at least one biomarker can be at least about 1.0, at least about 1.5, at least about 2.0, at least about 2.5, at least about 3.0, at least about 3.5, at least about 4.0, at least about 4.5, at least about 5.0, at least about 5.5, at least about 6.0, at least about 6.5, at least about 7.0, at least about 7.5, at least about 8.0, at least about 8.5, at least about 9.0, at least about 9.5, at least about 10.0, at least about 10.5, at least about 11.0, at least about 11.5, at least about 12.0, at least about 12.5, at least about 13.0, at least about 13.5, at least about 14.0, at least about 15.0, at least about 16.0, at least about 17.0, at least about 18.0, at least about 19.0, or at least about 20.0. In other embodiments, the fold change in the RNA expression level, relative to a control sample where the melanoma was without recurrence for at least about 5.0 years, in one or more of the at least one biomarker, can be no more than about −0.5, no more than about −1.0, no more than about −1.5, no more than about −2.0, no more than about −2.5, no more than about −3.0, no more than about −3.5, no more than about −4.0, no more than about −4.5, no more than about −5.0, or no more than about −6.0. In certain embodiments, the fold change in the RNA expression level, relative to a control sample where the melanoma was without recurrence for at least about 5.0 years, in one or more of the at least one biomarker, can be about −6.0, about −5.0, about −4.5, about −4.0, about −3.5, about −3.0, about −2.5, about −2.0, about −1.5, about −1.0, about −0.75, about −0.50, about −0.25, about 0.25, about 0.50, about 0.75, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.5, about 7.0, about 7.5, about 8.0, about 8.5, about 9.0, about 9.5, about 10.0, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20.0, about 20.5, from about −0.25 to about −6.0, from about −0.25 to about −2.0, from about −0.25 to about −1.0, from about 1.0 to about 20.5, from about 1.5 to about 15.0, from about 2.0 to about 15.0, or from about 2.5 to about 13.0.

The fold changes for microarray are calculated as described in the Statistical Analysis section of the Materials and Methods in the Examples described herein; see also Hao et al. (2017) “Sentinel lymph node genes to predict prognosis in node-positive melanoma patients” Ann Surg Oncol., Vol. 24, No. 1, pp. 108-116 and Menefee et al. (2020) “Age-related transcriptome changes in melanoma patients with tumor-positive sentinel lymph nodes” AGING, Vol. 12, No. 24, pp. 24914-24939. The fold changes for NanoString are calculated as described in the Statistical Analysis section of the Materials and Methods in the Examples described herein; see also Hao et al. (2017) “Sentinel lymph node genes to predict prognosis in node-positive melanoma patients” Ann Surg Oncol., Vol. 24, No. 1, pp. 108-116 and Menefee et al. (2020) “Age-related transcriptome changes in melanoma patients with tumor-positive sentinel lymph nodes” AGING, Vol. 12, No. 24, pp. 24914-24939. The fold changes for PCR are calculated with the 2^{-ΔΔCt} method; see also Hao et al. (2017) “Sentinel lymph node genes to predict prognosis in node-positive melanoma patients” Ann Surg Oncol., Vol. 24, No. 1, pp. 108-116 and Menefee et al. (2020) “Age-related transcriptome changes in melanoma patients with tumor-positive sentinel lymph nodes” AGING, Vol. 12, No. 24, pp. 24914-24939.

In some embodiments, the method further comprises assessing a clinicopathologic feature of the subject (e.g., human or primate) from which the sample was obtained. In certain embodiments, consideration of clinicopathologic features can in some cases increase specificity and sensitivity of the prognosis and/or the treatment. In other embodiments, the clinicopathologic feature of the subject (e.g., human) from which the sample was obtained and the clinicopathologic feature is age, gender, anatomic location, Breslow thickness, ulceration, or sentinel lymph node status, or a combination thereof. In certain embodiments, the clinicopathologic feature of the subject (e.g., human or primate) from which the sample was obtained and the clinicopathologic feature is metastasis, age, lesion site, tumor burden, number of positive nodes, ulceration, tumor thickness, or a combination thereof.

In some embodiments, the subject has stage III melanoma.

In some embodiments, treating the subject (e.g., human or primate) comprises administering to the subject (e.g., human) one or more of immunotherapy, interferon, a BRAF inhibitor (e.g., Proietti et al. (2020) “BRAF Inhibitors: Molecular Targeting and Immunomodulatory Actions” Cancers (Basel), Vol. 12, No. 7, Article 1823, 13 pages, which is herein incorporated by reference in its entirety), a checkpoint inhibitor (e.g., Darvin et al. (2018) “Immune checkpoint inhibitors: recent progress and potential biomarkers” Experimental & Molecular Medicine, Vol. 50, Article 165, 11 pages, which is herein incorporated by reference in its entirety), a Wnt10b inhibitor (e.g., Goldsberry et al. (2019) “A Review of the Role of Wnt in Cancer Immunomodulation” Cancers, Vol. 11, Article 771, 19 pages, which is herein incorporated by reference in its entirety), or an IRAK3 inhibitor (e.g., Singer et al. (2018) “Inhibition of interleukin-1 receptor-associated kinase 1 (IRAK1) as a therapeutic strategy” Oncotarget, Vol. 9, No. 70, pp. 33416-33439, which is herein incorporated by reference in its entirety; Hossen et al. (2017) “Thymoquinone: An IRAK1 inhibitor with in vivo and in vitro antiinflammatory activities” Scientific Reports, Vol. 7, Article 42995, 12 pages, which is herein incorporated by reference in its entirety). In certain embodiments, treating the subject comprises administering to the subject one or more of a Wnt10b inhibitor or an IRAK3 inhibitor. In some embodiments, treating the subject (e.g., human) comprises administering to the subject one or more of immunotherapy, interferon-gamma, interferon alfa-2b, a BRAF inhibitor, vemurafenib, dabrafenib, trametinib, encorafenib, a checkpoint inhibitor, a PD-1 inhibitor, nivolumab, pembrolizumab, cemiplimab, a PD-L1 inhibitor, atezolizumab, avelumab, durvalumab, a cytotoxic T-lymphocyte antigen 4 (CTLA-4) inhibitor, ipilimumab, a Wnt10b inhibitor, DKN-01, CGX1321, ETC 1922159, an IRAK3 inhibitor, pacritinib, or thymoquinone. In other embodiments, the treating comprises administering interferon-gamma, interferon alfa-2b, or both. In yet other embodiments, the treating comprises administering a BRAF inhibitor, vemurafenib, dabrafenib, trametinib, encorafenib, or a combination thereof. In still other embodiments, the treating comprises administering a checkpoint inhibitor, a PD-1 inhibitor, nivolumab, pembrolizumab, cemiplimab, a PD-L1 inhibitor, atezolizumab, avelumab, durvalumab, a cytotoxic T-lymphocyte antigen 4 (CTLA-4) inhibitor, ipilimumab, or a combination thereof. In certain embodiments, the treating comprises administering a Wnt10b inhibitor, DKN-01, CGX1321, ETC 1922159, or a combination thereof. In some embodiments, the treating comprises administering an IRAK3 inhibitor, pacritinib, thymoquinone, or a combination thereof. In certain embodiments, treating further comprises one or more of surgery, chemotherapy, radiation therapy, targeted therapy, or vaccine therapy.

In some embodiments, the treating comprises administering a Wnt10b inhibitor, DKN-01, CGX1321, ETC 1922159 or a combination thereof and the subject (e.g., human) is at least about 40 years old, at least about 50 years old, at least about 60 years old, at least about 70 years old, at least about 80 years old, about 35, about 40, about 42, about 44, about 45, about 46, about 48, about 50, about 51, about 52, about 53, about 54, about 55, about 56, about 57, about 58, about 59, about 60, about 61, about 62, about 63, about 64, about 65, about 66, about 67, about 68, about 69, about 70, about 72, about 74, about 75, about 76, about 78, about 80, about 85, or about 90 years old.

In some embodiments, the treating comprises administering an IRAK3 inhibitor, pacritinib, thymoquinone, or a combination thereof and the subject (e.g., human) is no more than about 30 years old, no more than about 40 years old, no more than about 50 years old, no more than about 60 years old, no more than about 70 years old, about 10, about 15, about 20 , about 25, about 30, about 35, about 40, about 42, about 44, about 45, about 46, about 48, about 50, about 51, about 52, about 53, about 54, about 55, about 56, about 57, about 58, about 59, about 60, about 61, about 62, about 63, about 64, about 65, about 66, about 67, about 68, about 69, about 70, about 72, about 74, about 75, about 76, about 78, or about 80 years old.

In some embodiments, the treating only occurs if the fold change in the RNA expression level, relative to a control sample where the melanoma was without recurrence for at least about 5.0 years, in one or more of the at least one biomarker is about −6.0, about −5.0, about −4.5, about −4.0, about −3.5, about −3.0, about −2.5, about −2.0, about −1.5, about −1.0, about −0.75, about −0.50, about −0.25, about 0.25, about 0.50, about 0.75, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.5, about 7.0, about 7.5, about 8.0, about 8.5, about 9.0, about 9.5, about 10.0, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20.0, about 20.5, from about −0.25 to about −6.0, from about −0.25 to about −2.0, from about −0.25 to about −1.0, from about 1.0 to about 20.5, from about 1.5 to about 15.0, from about 2.0 to about 15.0, or from about 2.5 to about 13.0.

In certain embodiments, the treating comprises administering a Wnt10b inhibitor, DKN-01, CGX1321, ETC 1922159, or a combination thereof, only occurs if (a) the fold change in the RNA expression level, relative to a control sample where the melanoma was without recurrence for at least about 5.0 years, in one or more of the at least one biomarker is about −6.0, about −5.0, about −4.5, about −4.0, about −3.5, about −3.0, about −2.5, about −2.0, about −1.5, about −1.0, about −0.75, about −0.50, about −0.25, about 0.25, about 0.50, about 0.75, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.5, about 7.0, about 7.5, about 8.0, about 8.5, about 9.0, about 9.5, about 10.0, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20.0, about 20.5, from about −0.25 to about −6.0, from about −0.25 to about −2.0, from about −0.25 to about −1.0, from about 1.0 to about 20.5, from about 1.5 to about 15.0, from about 2.0 to about 15.0, or from about 2.5 to about 13.0 and (b) the subject is at least about 40 years old, at least about 50 years old, at least about 60 years old, at least about 70 years old, at least about 80 years old, about 35, about 40, about 42, about 44, about 45, about 46, about 48, about 50, about 51, about 52, about 53, about 54, about 55, about 56, about 57, about 58, about 59, about 60, about 61, about 62, about 63, about 64, about 65, about 66, about 67, about 68, about 69, about 70, about 72, about 74, about 75, about 76, about 78, about 80, about 85, or about 90 years old.

In certain embodiments, the treating comprises administering of IRAK3 inhibitor, pacritinib, thymoquinone, or a combination thereof, only occurs if (a) the fold change in the RNA expression level, relative to a control sample where the melanoma was without recurrence for at least about 5.0 years, in one or more of the at least one biomarker is about −6.0, about −5.0, about −4.5, about −4.0, about −3.5, about −3.0, about −2.5, about −2.0, about −1.5, about −1.0, about −0.75, about −0.50, about −0.25, about 0.25, about 0.50, about 0.75, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.5, about 7.0, about 7.5, about 8.0, about 8.5, about 9.0, about 9.5, about 10.0, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20.0, about 20.5, from about −0.25 to about −6.0, from about −0.25 to about −2.0, from about −0.25 to about −1.0, from about 1.0 to about 20.5, from about 1.5 to about 15.0, from about 2.0 to about 15.0, or from about 2.5 to about 13.0 and (b) the subject is no more than about 30 years old, no more than about 40 years old, no more than about 50 years old, no more than about 60 years old, no more than about 70 years old, about 10, about 15, about 20, about 25, about 30, about 35, about 40, about 42, about 44, about 45, about 46, about 48, about 50, about 51, about 52, about 53, about 54, about 55, about 56, about 57, about 58, about 59, about 60, about 61, about 62, about 63, about 64, about 65, about 66, about 67, about 68, about 69, about 70, about 72, about 74, about 75, about 76, about 78, or about 80 years old.

In some embodiments, the method comprising quantifying an RNA expression level for at least one biomarker in a sample from a sentinel lymph node (SLN) of the human, where the at least one biomarker comprises one or more of FOS, NR4A, or Wnt10b, and treating the human with a Wnt10b inhibitor, DKN-01, CGX1321, ETC 1922159, or a combination thereof, and optionally one or more of immunotherapy, interferon, a BRAF inhibitor, a checkpoint inhibitor, or an IRAK3 inhibitor. In certain aspects, the human is at least about 40 years old, at least about 50 years old, at least about 60 years old, at least about 70 years old, at least about 80 years old, about 35, about 40, about 42, about 44, about 45, about 46, about 48, about 50, about 51, about 52, about 53, about 54, about 55, about 56, about 57, about 58, about 59, about 60, about 61, about 62, about 63, about 64, about 65, about 66, about 67, about 68, about 69, about 70, about 72, about 74, about 75, about 76, about 78, about 80, about 85, or about 90 years old and the treating only occurs if the fold change in the RNA expression level, relative to a control sample where the melanoma was without recurrence for at least about 5.0 years, in one or more of the at least one biomarker is about −6.0, about −5.0, about −4.5, about −4.0, about −3.5, about −3.0, about −2.5, about −2.0, about −1.5, about −1.0, about −0.75, about −0.50, about −0.25, about 0.25, about 0.50, about 0.75, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.5, about 7.0, about 7.5, about 8.0, about 8.5, about 9.0, about 9.5, about 10.0, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20.0, about 20.5, from about −0.25 to about −6.0, from about −0.25 to about −2.0, from about −0.25 to about −1.0, from about 1.0 to about 20.5, from about 1.5 to about 15.0, from about 2.0 to about 15.0, or from about 2.5 to about 13.0.

In other embodiments, the method for treating melanoma in a human comprises quantifying an RNA expression level for at least one biomarker in a sample from a sentinel lymph node (SLN) of the human, where the at least one biomarker comprises IRAK3 and administering to the human an IRAK3 inhibitor, pacritinib, thymoquinone, or a combination thereof, and optionally one or more of immunotherapy, interferon, a BRAF inhibitor, a checkpoint inhibitor, or a Wnt10b inhibitor. In certain aspects of this methods, the human is no more than about 30 years old, no more than about 40 years old, no more than about 50 years old, no more than about 60 years old, no more than about 70 years old, about 10, about 15, about 20, about 25, about 30, about 35, about 40, about 42, about 44, about 45, about 46, about 48, about 50, about 51, about 52, about 53, about 54, about 55, about 56, about 57, about 58, about 59, about 60, about 61, about 62, about 63, about 64, about 65, about 66, about 67, about 68, about 69, about 70, about 72, about 74, about 75, about 76, about 78, or about 80 years old, and the treating only occurs if the fold change in the RNA expression level, relative to a control sample where the melanoma was without recurrence for at least about 5.0 years, in one or more of the at least one biomarker is about −6.0, about −5.0, about −4.5, about −4.0, about −3.5, about −3.0, about −2.5, about −2.0, about −1.5, about −1.0, about −0.75, about −0.50, about −0.25, about 0.25, about 0.50, about 0.75, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.5, about 7.0, about 7.5, about 8.0, about 8.5, about 9.0, about 9.5, about 10.0, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20.0, about 20.5, from about −0.25 to about −6.0, from about −0.25 to about −2.0, from about −0.25 to about −1.0, from about 1.0 to about 20.5, from about 1.5 to about 15.0, from about 2.0 to about 15.0, or from about 2.5 to about 13.0.

The presently-disclosed subject matter is further illustrated by the following specific but non-limiting examples. The following examples may include compilations of data that are representative of data gathered at various times during the course of development and experimentation related to the present invention.

### EXAMPLES

Menefee et al. (2020) “Age-related transcriptome changes in melanoma patients with tumor-positive sentinel lymph nodes” AGING, Vol. 12, No. 24, pp. 24914-24939, is herein incorporated by reference in its entirety. WO 2013/172947 A1 to Hao et al. is herein incorporated by reference in its entirety. US Pat. Appl. No. 2021/0010090 A1 to Hao et al. is herein incorporated by reference in its entirety.

### Materials and Methods

### Patient Selection

This study used two different technologies in three independent datasets of RNA samples obtained from melanoma patients with positive SLNs to identify age-related transcriptome changes in SLN and their association with outcome.

Microarray analysis was performed in the first independent dataset to assess 97 samples obtained from the Sunbelt Melanoma Trial (SMT). The samples were randomly chosen from among 317 melanoma patients with positive SLNs. This patient cohort has been described previously (Hao et al. (2017) “Sentinel lymph node genes to predict prognosis in node-positive melanoma patients” Ann Surg Oncol., Vol. 24, No. 1, pp. 108-116, which is herein incorporated by reference in its entirety). Thirty-nine patients experienced recurrence melanoma in this cohort, and fifty-eight patients did not experience recurrence. Median follow-up was 93 months. This study was approved by the institutional review boards (IRB) of each participating institution. Clinicopathologic factors, recurrence, and survival data were collected prospectively. Additional details of the SMT are described elsewhere (Hao et al. (2017) “Sentinel lymph node genes to predict prognosis in node-positive melanoma patients” Ann Surg Oncol., Vol. 24, No. 1, pp. 108-116); McMasters et al. (2016)

“Final results of the Sunbelt Melanoma Trial: a multi-institutional prospective randomized phase III study evaluating the role of adjuvant high dose interferon alfa-2b and completion lymph node dissection for patients staged by sentinel lymph node biopsy” J Clin Oncol., Vol. 34, pp. 1079-1086).

NanoString analysis was applied to the second patient cohort, which included 12 patients with tumor-positive SLNs from the James Graham Brown Cancer Center Biorepository at University of Louisville. This study followed an approved IRB protocol. There were 6 patients who experienced recurrence (3 of each at age <60 and ≥60 years old) and 6 patients who did not experience recurrence (3 of each at age <60 and ≥60 years old). Median follow-up was 34 months.

The third independent dataset of 36 samples from the James Graham Brown Cancer Center Biorepository was used to validate the differentially expressed genes (DEGs). The SLN tissue was acquired from patients at the time of surgical treatment of cutaneous melanoma, including staging with SLN biopsy between 2003 and 2017. Median follow-up of this cohort was 33.2 months. Patient characteristics such as age and outcome from all three datasets are summarized in Supplementary Table 7.

Supplementary Table 7

| | 1^{st} microarray | | 2^{nd} NanoString | | 3^{rd} dataset | |
|---|---|---|---|---|---|---|
| | dataset | | dataset | | qRT-PCR | |
| Outcome | <60 | ≥60 | <60 | ≥60 | <60 | ≥60 |
| No recurrence (recur^{no}) | 51 | 7 | 3 | 3 | 9 | 13 |
| Recurrence (recur^{yes}) | 28 | 11 | 3 | 3 | 9 | 5 |

### Definition of Age Groups

To ensure that we had a large enough sample size for a robust analysis, we grouped patients into two age groups. Patients were defined as being older if they were >60 years old (yr⁶⁰⁺). Patients were defined as being younger if they were <60 years old (yr⁶⁰⁻).

### Microarray Experiments

GeneChip Human HG-U133 plus 2.0 array (Affymetrix, Santa Clara, CA) was used in the first microarray dataset according to the manufacturer's guidelines. Details of RNA isolation, microarray experiment, and quality control were described in detail previously (Hao et al. (2017) “Sentinel lymph node genes to predict prognosis in node-positive melanoma patients” Ann Surg Oncol., Vol. 24, No. 1, pp. 108-116). This set of microarray data is accessible through NCBI's Gene Expression Omnibus (GEO, <<www.ncbi.nlm nih.gov/geo>>) by accession number GSE 43081.

NanoString Analysis of mRNA Expression of Immune Panel Genes and Immune Pathway Panel Genes

The second dataset of 12 RNA samples were isolated from fresh-frozen human SLN tissues from melanoma patients using RNeasy Plus Mini Kit (Qiagen). RNA quality control/quantity assessment (QC/QA) was checked by Agilent bioanalyzer. The RNA concentration was measured by Qubit. Total RNA (100 ng per sample) were analyzed on the nCounter MAX system. Two gene expression assays were used: PanCancer immune profiling and PanCancer immune pathway profiling (NanoString Technologies, Seattle, WA, USA). PanCancer immune profiling assay comprised 730 immune-related genes and 40 internal reference genes Immune pathway profiling assay comprised 730 genes from 13 canonical pathways and 40 selected reference genes. Raw counts for each assay were collected using the NanoString data analysis software (nSolver).

Quantitative Reverse Transcriptase Polymerase Chain Reaction (qRT-PCR)

The third dataset of 36 RNA samples were isolated from fresh-frozen human SLN tissues from melanoma patients using RNeasy Plus Mini Kit (Qiagen). Total SLN RNA (1000 ng) from each sample was reverse-transcribed with the SuperScript III First-Strand Synthesis System. mRNA primers were purchased from Life Technologies (Carlsbad, CA). Quantitative RT-PCR reactions were completed on a 7500 Fast Real Time PCR system (Life Technologies). The relative quantity of the target mRNA was normalized to endogenous gene (B2M). The fold changes (FC) of each mRNA in the qRT-PCR experiments were calculated with the 2^{-ΔΔCt} method.

### Statistical Analysis

For microarray analysis, a fold change outlier (FCO) filter was applied independently to reduce the dimension of the data before determining the DEGs between the two age groups (yr⁶⁰⁺ and yr⁶⁰⁻) as well as between patients with recurrence (recur^{yes}) and those without recurrence (recur^{no}) (Bolstad et al. (2003) “A comparison of normalization methods for high density oligonucleotide array data based on variance and bias” Bioinformatics, Vol. 19, pp. 185-193; Tusher et al. (2001) “Significance analysis of microarrays applied to the ionizing radiation response” Proc Natl Acad Sci USA., Vol. 98, pp. 5116-5121). For each of 54,675 probes on the array, the fold change (FC) was calculated and four filters (T1, T2, T3 and T4) were used. T1={μ(FC)±1.5σ(FC)}, T2={μ(FC)±2σ(FC)}, T3={μ(FC)±3σ(FC)}, and T4={μ(FC)±4σ(FC)}, where μ(FC) is the mean of fold changes (FC) and σ is the standard deviation of FC from all 54,675 probes in the array. The genes that fell inside T1, T2, and T3 were filtered from the differential data. After filtering the data, a t-test for normal gene expression data and a Wilcoxon test for non-normal expression data were applied (Khan (2005) “ArrayVigil: a methodology for statistical comparison of gene signatures using segregated-one-tailed (SOT) Wilcoxon's signed-rank test” J Mol Biol., Vol. 345, pp. 645-649). The Benjamini-Hochberg method was employed to adjust the p values (Benjamini et al. (1995) “Controlling the false discovery rate: a practical and powerful approach to multiple testing” J R Statis Soc B., Vol. 57, pp. 289-300). When comparing the changes of the SLN gene expressions in the yr⁶⁰⁺ versus yr⁶⁰⁻ patients, a multivariable linear regression model was fitted for each gene about age (<60 or ≥60). The equation used is below:

Gene Expression=α+β1 age, where age=1 if≥60 years old and 0 otherwise.

The estimates and p values are presented by filter T2, T3, and T4. When assessing the changes of the SLN gene expressions that are associated with recur^{yes} versus recur^{no} in the yr⁶⁰⁺ and yr⁶⁰⁻ melanoma patients, a multivariable linear regression model was fitted for each gene of each sample about age (<60 years or ≥60), outcome (recur^{yes} or recur^{no}), and the interaction of age and outcome. The equation used is below:

Gene Expression=α+β1 age+β2 outcome+β3 age*outcome, where age=1 if≥60 years old and 0 otherwise, outcome=1 if recur^{yes} and 0 otherwise.

The estimate and p values are also presented by filter T2, T3, and T4. Statistical Analysis System (SAS) was used to perform the regression analysis. p values of FC were calculated using ANOVA (Cary NC. (2003) The SAS system V9. Cary, NC: SAS Institute Inc.; Gonen (2006) “Receiver operating characteristics (ROC) curves” In: Proceedings of the thirty-first annual SAS users group international conference, pp. 210-231).

For the NanoString results analysis, positive control normalization was performed by using gene expression data normalized to the mean of the positive control probes for each assay. RNA content normalization was performed by using gene expression data normalized to the geometric mean of housekeeping genes in the CodeSet. Raw data are also analyzed using the nSolver Advanced Analysis module. More information on the Advanced Analysis package can be found at <<www.nanostring.com/products/nSolver>>.

Ingenuity Pathway Analysis (IPA) software (Ingenuity Systems, Redwood City, CA) was used for gene network and pathway analysis. The statistical score of a pathway is defined as —log (p value) from Fisher's exact test analysis.

### ABBREVIATIONS

CI Confidence interval
DEGs Differentially expressed genes
FC Fold changes
FCO Fold change outlier
FOS FBJ murine osteosarcoma viral oncogene homolog
GEO Gene Expression Omnibus
IRAK3 Interleukin-1 receptor-associated kinase 3
ITGB1 Integrin subunit beta 1
ITGBL1 Integrin subunit beta like 1
NR4A2 Nuclear receptor subfamily 4, group A, member 2
PPAR Peroxisome proliferator-activated receptor
QC/QA Quality control/quantity assessment
qRT-PCR Quantitative reverse transcriptase polymerase chain reaction
recur^{no} Without recurrence
recur^{yes} Recurrence
SLN Sentinel lymph node
SMT Sunbelt melanoma Trial
TERT Telomerase reverse transcriptase
Tregs T regulatory cells
yr⁶⁰⁻ <60 years old
yr⁶⁰⁺ ≥60 years old

### Results

### Transcriptome Changes in SLN Genes in Older Patients (≥60 Years Old) Versus Younger Patients (<60 Years Old) by Microarray Analysis

We were interested in comparing gene expression profiles in older versus younger patients and in assessing whether there was a correlation with melanoma recurrence. Therefore, we analyzed the first microarray dataset from 97 melanoma patients with positive SLNs from the Sunbelt Melanoma Trial (SMT) and evaluated the transcriptome changes of the SLN by two defined age groups: the older and the younger groups. Patients were defined as being older if they were ≥60 years old (yr⁶⁰⁺). Patients were defined as being younger if they were <60 years old (yr⁶⁰⁻).

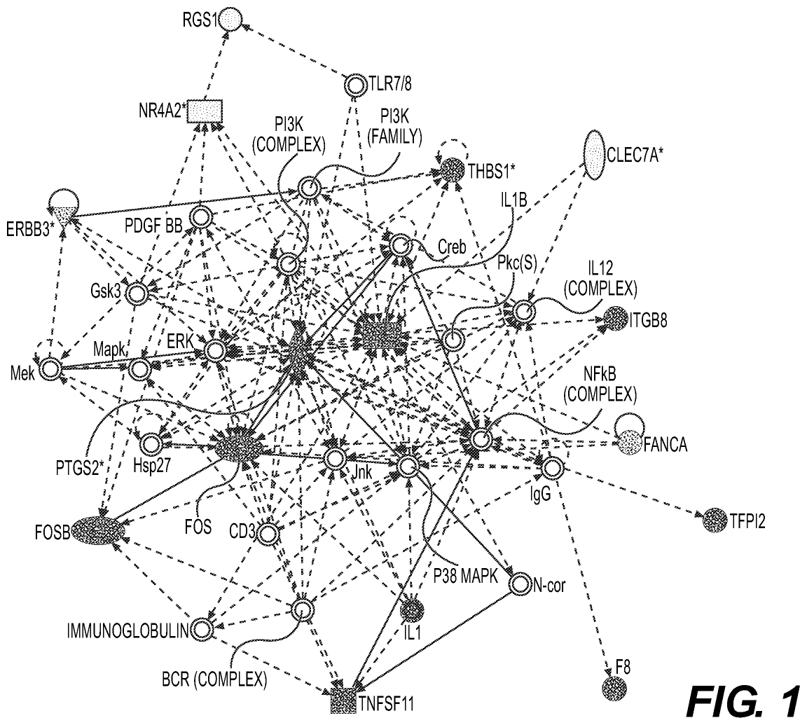

Table 1 lists the clinical data of the 97 melanoma patients grouped by age. In this dataset, there were no significant differences between the two age groups in primary tumor site, Breslow thickness, Clark level, or ulceration presence. However, in younger patients, the recurrence rate was significantly higher when Breslow thickness was higher. In older patients, there were no significant differences in Breslow thickness, Clark level, and ulceration presence between groups of patients with recurrence (recur^{yes}) and those without recurrence (recur^{no}). Using microarray filter T3 and T4, we detected a total of 577 and 156 differentially expressed probe sets, in older versus the younger patients. Among them, there were 41 and 11 differentially expressed probe sets by filters T3 and T4 in the older versus younger groups (p<0.05). Probe sets without defined gene names by annotation from Partek Genomics Suite software were removed from the lists. There were 7 differentially expressed genes (DEGs) in the yr⁶⁰⁺ group versus the yr⁶⁰⁻ group with a p value <0.05 by T4 filter (Table 2). Among them, 1-13J murine osteosarcoma viral oncogene homolog (FOS) and nuclear receptor subfamily 4, group A, member 2 (NR4A2), were the two genes that had significant higher expression in the yr⁶⁰⁺ group than in the yr⁶⁰⁻ group. The DEGs between the yr⁶⁰⁻ and the yr⁶⁰⁺ group had various biological functions, including toll-like receptor signaling pathway transduction, adaptive and innate immune response, autophagy, and transcription regulation (Table 2). The network connection of the 156 DEGs by T4 filter is shown in FIG. 1. The top canonical pathway that showed a difference in the yr⁶⁰⁻ and the yr⁶⁰⁺ group was the peroxisome proliferator-activated receptor (PPAR) signaling pathway, which had a close interaction with toll-like receptor signaling pathway (Supplementary Table 1) (Dana et al. (2020) “The effect of fenofibrate, a PPARα activator on toll-like receptor-4 signal transduction in melanoma both in vitro and in vivo” Clin Transl Oncol., Vol. 22, No. 4, pp. 486-494 ; Dana et al. (2019) “PPARγ agonist, pioglitazone, suppresses melanoma cancer in mice by inhibiting TLR4 signaling” J Pharm Pharm Sci., Vol. 22, pp. 418-423). The list of all DEGs by T3 filter is listed in Supplementary Table 2.

TABLE 1
Clinical data of the first dataset (97 melanoma patients) grouped by age

| | | | | | | | P Value |
|---|---|---|---|---|---|---|---|
| | Age ≤ 60 | | | Age > 60 | | | for |
| | No | | | No | | | Age < 60 |
| | recurrence | Recurrence | P | recurrence | Recurrence | P | vs |
| Variables | (N = 51) | (N = 28) | Value | (N = 7) | (N = 11) | Value | Age ≥ 60 |
| Gender | | | 0.957 | | | 1.000 ^{†} | 0.541 |
| Female (%) | 24 | 13 | | 3 | 4 | | |
| | (47.1) | (46.4) | | (42.9) | (36.4) | | |
| Male (%) | 27 | 15 | | 4 | 7 | | |
| | (52.9) | (53.6) | | (57.1) | (63.6) | | |
| Primary Site | | | 0.908 ^{†} | | | 0.141 ^{†} | 0.371 ^{†} |
| Head (%) | 2 | 1 | | | | | |
| | (3.9) | (3.6) | | | | | |
| Lower Extremity | 12 | 9 | | 2 | 5 | | |
| (%) | (23.5) | (32.1) | | (28.6) | (45.5) | | |
| Neck (%) | 1 | 0 | | 0 | 1 | | |
| | (2.0) | (0.0) | | (0.0) | (9.1) | | |
| Trunk (%) | 28 | 13 | | 4 | 2 | | |
| | (54.9) | (46.4) | | (57.1) | (18.2) | | |
| Upper Extremity | 8 | 5 | | 0 | 3 | | |
| (%) | (15.7) | (17.9) | | (0.0) | (27.3) | | |
| Breslow | | | 0.006 | | | 0.837 | 0.362 |
| Thickness (mm) | | | | | | | |
| Mean (95% CI) | 2.5 | 3.9 | | 2.6 | 2.5 | | |
| | (2.2-2.9 | (2.8-5.0) | | (1.2-4.1) | (1.8-3.1) | | |
| Median (min- | 2.0 | 2.7 | | 2.5 | 2.4 | | |
| max) | (1.0-6.0) | (1.5-13.0) | | (1.2-6.8) | (1.1-4.4) | | |
| Clark level | | | 0.741 ^{†} | | | 1.000 ^{†} | 0.457 ^{†} |
| II/III (%) | 7 | 3 | | 2 | 2 | | |
| | (13.7) | (10.7) | | (28.6) | (18.2) | | |
| IV/V (%) | 43 | 25 | | 5 | 9 | | |
| | (84.3) | (89.3) | | (71.4) | (81.8) | | |
| Ulceration | | | 0.255 ^{†} | | | 0.430 ^{†} | 0.268 ^{†} |
| Present | | | | | | | |
| NA (%) | 0 | 1 | | 1 | 0 | | |
| | (0.0) | (3.6) | | (14.3) | (0.0) | | |
| No (%) | 34 | 15 | | 3 | 5 | | |
| | (66.7) | (53.6) | | (42.9) | (45.5) | | |
| Yes (%) | 17 | 12 | | 2 | 6 | | |
| | (33.3) | (42.9) | | (28.6) | (54.5) | | |
| Time To FU | | | <.001 | | | 0.015 | 0.187 |
| (All Patients) | | | | | | | |
| Mean (95% CI) | 86.8 | 65.5 | | 88.7 | 57.3 | | |
| | (80.8-92.7) | (53.3-77.7) | | (73.0-104.5) | (42.3-72.2) | | |
| Median (min- | 92.0 | 58.5 | | 94.0 | 57.0 | | |
| max) | (40.0-122.0) | (6.0-122.0) | | (51.0-111.0) | (16.0-111.0) | | |

TABLE 2
The DEGs in the SLN in yr⁶⁰⁺ versus yr⁶⁰⁻ patients in
the microarray dataset using T4 filter (P < 0.05).

| Gene | | | P | Fold |
|---|---|---|---|---|
| symbol | Gene Name | Biological function | value | change |
| FOSB | FBJ murine | negative regulation of transcription | 0.021 | 1.60 |
| | osteosarcoma | from RNA polymerase II promoter | | |
| | viral oncogene | | | |
| | homolog B | | | |
| FOS | FBJ murine | toll-like receptor signaling | 0.0255 | 1.56 |
| | osteosarcoma | pathway//MyD88-dependent and - | | |
| | viral oncogene | independent toll-like receptor | | |
| | homolog | signaling pathway | | |
| NR4A2 | nuclear receptor | negative regulation of transcription | 0.0096 | 1.47 |
| | subfamily 4, | from RNA polymerase II | | |
| | group A, member | promoter//response to hypoxia | | |
| | 2 | | | |
| CLEC4C | C-type lectin | stimulatory C-type lectin receptor | 0.049 | 1.45 |
| | domain family 4, | signaling pathway//adaptive and | | |
| | member C | innate immune response | | |
| LIX1 | limb and CNS | autophagy//autophagosome | 0.0098 | 1.41 |
| | expressed 1 | maturation | | |
| NRCAM | neuronal cell | angiogenesis//neuron migration/cell | 0.0008 | 1.40 |
| | adhesion | adhesion | | |
| | molecule | | | |
| GRB14 | growth factor | signal transduction | 0.0135 | 0.79 |
| | receptor bound | | | |
| | protein 14 | | | |

SUPPLEMENTARY TABLE 1
Top canonical pathways that showed differences in yr⁶⁰⁺ versus
yr⁶⁰⁻ patients in the first microarray dataset using T4 filter.

| Pathway name | p-value | Overlap | |
|---|---|---|---|
| PPAR Signaling | 2.65E−04 | 4.0% | (4/101) |
| Acute phase response signaling | 2.20E−03 | 2.2% | (4/178) |
| Melanocyte development and | 3.13E−03 | 3.2% | (3/95) |
| pigmentation signaling | | | |
| Coagulation system | 5.18E−03 | 5.7% | (2/35) |
| Cholecystokinin/Gastrin-mediated | 5.61E−03 | 2.6% | (3/117) |
| signaling | | | |

SUPPLEMENTARY TABLE 2
Differentially expressed genes (DEGs) in the SLN in yr⁶⁰⁺ versus
yr⁶⁰⁻ patients in the first microarray dataset using T3 filter.

| | | P | Fold |
|---|---|---|---|
| Gene symbol | Gene Name | value | change |
| FOSB | FBJ murine osteosarcoma viral oncogene homolog B | 0.0208 | 1.5961 |
| FOS | FBJ murine osteosarcoma viral oncogene homolog | 0.0255 | 1.5558 |
| DUSP1 | dual specificity phosphatase 1 | 0.0453 | 1.5349 |
| NR4A2 | nuclear receptor subfamily 4, group A, member 2 | 0.0096 | 1.4719 |
| IDO1 | indoleamine 2,3-dioxygenase 1 | 0.0155 | 1.454 |
| CLEC4C | C-type lectin domain family 4, member C | 0.0486 | 1.4497 |
| LIX1 | limb and CNS expressed 1 | 0.0098 | 1.4118 |
| CD8A | CD8a molecule | 0.0003 | 1.4111 |
| BACH2 | BTB and CNC homology 1, basic leucine zipper | 0.003 | 1.3963 |
| | transcription factor 2 | | |
| NRCAM | neuronal cell adhesion molecule | 0.0008 | 1.3961 |
| NOG | noggin | 0.0027 | 1.3839 |
| KLRC4- | KLRC4-KLRK1 read through /// killer cell lectin-like | 0.0011 | 1.3827 |
| KLRK1 /// | receptor subfamily K, member 1 | | |
| KLRK1 | | | |
| MS4A6A | membrane-spanning 4-domains, subfamily A, member 6A | 0.0434 | 1.3709 |
| KLF4 | Kruppel-like factor 4 (gut) | 0.0278 | 1.3662 |
| SATB1 | SATB homeobox 1 | 0.0322 | 1.3484 |
| LOC101928963 | uncharacterized LOC101928963 | 0.027 | 1.2614 |
| GRIK2 | glutamate receptor, ionotropic, kainate 2 | 0.0497 | 0.8748 |
| MUC15 | mucin 15, cell surface associated | 0.0495 | 0.8681 |
| DLK1 | delta-like 1 homolog (Drosophila) | 0.0339 | 0.8617 |
| RNF152 | ring finger protein 152 | 0.05 | 0.8483 |
| ITGBL1 | integrin beta like 1 | 0.03 | 0.8466 |
| ERGIC3 | ERGIC and golgi 3 | 0.0295 | 0.8456 |
| INHBA | inhibin beta A | 0.0461 | 0.8347 |
| PRUNE2 | prune homolog 2 (Drosophila) | 0.0059 | 0.8323 |
| LINC00354 | long intergenic non-protein coding RNA 354 | 0.0292 | 0.8312 |
| MKX | mohawk homeobox | 0.0053 | 0.8308 |
| WWC1 | WW and C2 domain containing 1 | 0.0295 | 0.8228 |
| LOC105373225 | uncharacterized LOC105373225 | 0.029 | 0.8139 |
| SLC13A5 | solute carrier family 13 (sodium-dependent citrate | 0.018 | 0.7921 |
| | transporter), member 5 | | |
| GRB14 | growth factor receptor bound protein 14 | 0.0135 | 0.792 |
| ATP2B2 | ATPase, Ca++ transporting, plasma membrane 2 | 0.0295 | 0.791 |
| COL28A1 | collagen, type XXVIII, alpha 1 | 0.0092 | 0.7779 |
| LOC100507516 | uncharacterized LOC100507516 | 0.0223 | 0.7719 |
| MLANA | melan-A | 0.0486 | 0.7057 |

### Transcriptome Changes of Immune Genes and Immune Pathway Genes in the SLNs of Older Versus Younger Patients as Assessed by NanoString Analysis

Immune cells are a component of lymph node structure. We then focused on immune genes and immune pathways associated with both age groups and assessed by NanoString analysis. This analysis in the second dataset found that 12 immune-related genes were differentially expressed in SLNs in older versus younger patients (Table 3). There were 17 immune pathway-related genes in SLNs that were differentially expressed in yr⁶⁰⁺ versus yr⁶⁰⁻ patients (Table 4). Of note is that the NR4A2 gene was found to be differentially expressed in yr⁶⁰⁺ versus yr⁶⁰⁻ patients from the first microarray dataset. The NR4A3 gene, which belongs to the same family members of NR4A2, was also found to have a higher fold change (FC) in yr⁶⁰⁺ patients, the p value is 0.0517 (last row in Table 4). The immune gene, integrin subunit beta 1 (ITGB1), was found to be differentially expressed in yr⁶⁰⁺ versus the yr⁶⁰⁻ patients (Table 3). Integrin subunit beta like 1 (ITGBL1) was also found to be differentially expressed in yr⁶⁰⁺ versus yr⁶⁰⁻ patients by microarray analysis (Supplementary Table 2). The immune gene with the highest and lowest fold change in the yr⁶⁰⁺ versus the yr⁶⁰⁻ patient group was melanoma antigen family A, 3 (MAGEA3) and leukemia inhibitory factor (LIF) (fold change=2.87 and −1.16) (Table 3). Among the three highest fold changes of the immune pathway genes, two of them were secreted frizzled-related protein 2 and 4 (SFRP2 and SFRP4) (fold change=1.93 and 1.78) (Table 4). Both genes belong to the Wnt pathway.

NanoString results suggested that NR4A and ITGB1 genes are more highly expressed immune genes in older melanoma patients compared to their younger counterparts with lymph node metastasis. These genes, therefore, might be responsible for the age-related differences in response of SLN to the presence of nodal metastasis. The Wnt pathway might also be a relevant immune pathway associated with age-related immune response to melanoma metastasis to the SLN.

TABLE 3
Immune genes that were differentially expressed in the SLN in yr⁶⁰⁺ versus
yr⁶⁰⁻ patients in the second dataset by NanoString analysis (P < 0.05).

| | | | Fold |
|---|---|---|---|
| Gene symbol | Gene Name | P value | change |
| MAGEA3 | melanoma antigen family A, 3 | 0.0149 | 2.87 |
| MME | membrane metallo-endopeptidase | 0.0466 | 1.34 |
| CD244 | CD244 molecule, natural killer cell receptor 2B4 | 0.0453 | 1.07 |
| CDKN1A | cyclin-dependent kinase inhibitor 1A (p21, Cip1) | 0.0254 | 0.792 |
| JAM3 | junctional adhesion molecule 3 | 0.0405 | 0.628 |
| ITGB1 | integrin subunit beta 1 | 0.00565 | 0.564 |
| ALCAM | activated leukocyte cell adhesion molecule | 0.00272 | 0.489 |
| MAVS | mitochondrial antiviral signaling protein | 0.00886 | 0.303 |
| IFIH1 | interferon induced with helicase C domain 1 | 0.0118 | −0.325 |
| MX1 | myxovirus (influenza virus) resistance 1, | 0.0493 | −0.666 |
| | interferon-inducible protein p78 (mouse) | | |
| CXCL3 | chemokine (C-X-C motif) ligand 3 | 0.0393 | −0.851 |
| LIF | leukemia inhibitory factor | 0.0476 | −1.16 |

TABLE 4
Immune pathway genes that are differentially expressed in the SLN in yr⁶⁰⁺
versus yr⁶⁰⁻ patients in the second dataset by NanoString analysis (P < 0.05)*.

| | | | Fold |
|---|---|---|---|
| Gene symbol | Gene Name | P value | change |
| COMP | cartilage oligomeric matrix protein | 0.0212 | 2.51 |
| SFRP2 | secreted frizzled-related protein 2 | 0.0428 | 1.93 |
| SFRP4 | secreted frizzled-related protein 4 | 0.0279 | 1.78 |
| CTNNB1 | catenin (cadherin-associated protein), beta 1, | 0.0247 | 0.832 |
| | 88 kDa | | |
| CDKN1A | cyclin-dependent kinase inhibitor 1A (p21, Cip1) | 0.0345 | 0.702 |
| PLD1 | phospholipase D1, phosphatidylcholine-specific | 0.0088 | 0.661 |
| TNC | tenascin C | 0.0207 | 0.655 |
| PBRM1 | polybromo 1 | 0.00195 | 0.463 |
| GADD45A | growth arrest and DNA-damage-inducible, alpha | 0.00483 | 0.421 |
| FUT8 | fucosyltransferase 8 (alpha (1,6) fucosyltransferase) | 0.0394 | 0.367 |
| PIK3R3 | phosphoinositide-3-kinase, regulatory subunit 3 | 0.0278 | 0.317 |
| | (gamma) | | |
| PRKAR2A | protein kinase, cAMP-dependent, regulatory, type | 0.0167 | 0.296 |
| | II, alpha | | |
| PIK3CB | phosphatidylinositol-4,5-bisphosphate 3-kinase, | 0.0454 | 0.283 |
| | catalytic subunit beta | | |
| FANCB | Fanconi anemia, complementation group B | 0.0385 | −0.589 |
| ERCC2 | excision repair cross-complementing rodent repair | 0.0176 | −0.595 |
| | deficiency, complementation group 2 | | |
| TNFRSF10C | tumor necrosis factor receptor superfamily, | 0.0123 | −0.677 |
| | member 10c, decoy without an intracellular domain | | |
| CDK6 | cyclin-dependent kinase 6 | 0.0308 | −0.683 |
| NR4A3 | nuclear receptor subfamily 4, group A, member 3 | 0.0517 | 1.3 |

Transcriptome Changes in SLN Associated with Recurrence in yr⁶⁰⁺ or yr⁶⁰⁻ Melanoma Patients by Microarray Analysis

After we compared the transcriptome changes in SLN genes between the yr⁶⁰⁺ and the yr⁶⁰⁻ melanoma patients, we studied whether there were any differences between patients who experienced recurrence versus those who remained disease free. We also evaluated these results by age categories. A multivariable linear regression model was fitted for each gene of each sample about age (yr⁶⁰⁺ or yr⁶⁰⁻), outcome (recur^{yes} or recur^{no}), and the interaction of age and outcome in the first microarray dataset. There were 100 differentially expressed probe sets with a statistically significant difference (p<0.05) after adjusting either by age or outcome or the interaction of age and outcome using filter T4. Among them, there were 11 differentially expressed probe sets with a significant difference adjusting by the interaction of age and outcome (p<0.05). Probe sets of the same gene were merged. There were 6 genes with statistically significant differences between groups (Table 5). We further analyzed the mean and 95% confidence interval (CI) of these 6 DEGs (Table 5). Means (95% CI) without overlapped values between each group were italicized. The non-overlapped values implied that there were statistically significant differences between the two groups. For example, NR4A2 was differentially expressed in yr⁶⁰⁺ versus yr⁶⁰⁻ melanoma patients without recurrence. NR4A2 also showed differences in yr⁶⁰⁺ patients with (recur^{yes}) versus those without recurrence (recur^{no}) (Table 5).

TABLE 5
Mean and 95% confidence interval (CI) of the DEGs adjusted
by the interaction of age and outcome using a multivariable
linear regression model in the first microarray dataset.

| | | Young (<60 years old) | | Old (≥60 years old) | | |
|---|---|---|---|---|---|---|
| | | No | | No | | |
| | Total | recurrence | Recurrence | recurrence | Recurrence | P |
| Variables | (N = 97) | (N = 51) | (N = 28) | (N = 7) | (N = 11) | Value |
| NR4A2 | | | | | | <.001 |
| Mean | 6.0 | 6.0 | 5.6 | 7.6 | 5.7 | |
| (95% CI) | (5.8-6.2) | (5.8-6.2) | (5.5-5.8) | (6.4-8.8) | (5.5-6.0) | |
| Mean ± SE | 6.0 ± 0.1 | 6.0 ± 0.1 | 5.6 ± 0.1 | 7.6 ± 0.6 | 5.7 ± 0.1 | |
| IL1B | | | | | | <.001 |
| Mean | 6.5 | 6.5 | 6.2 | 7.8 | 6.0 | |
| (95% CI) | (6.3-6.6) | (6.3-6.7) | (5.9-6.5) | (6.6-9.0) | (5.7-6.2) | |
| Mean ± SE | 6.5 ± 0.1 | 6.5 ± 0.1 | 6.2 ± 0.1 | 7.8 ± 0.6 | 6.0 ± 0.1 | |
| TFPI2 | | | | | | <.001 |
| Mean | 5.5 | 5.7 | 5.4 | 6.4 | 4.7 | |
| (95% CI) | (5.4-5.7) | (5.5-5.9) | (5.1-5.8) | (5.8-7.0) | (4.2-5.1) | |
| Mean ± SE | 5.5 ± 0.1 | 5.7 ± 0.1 | 5.4 ± 0.2 | 6.4 ± 0.3 | 4.7 ± 0.2 | |
| CLEC7A | | | | | | 0.004 |
| Mean | 4.7 | 4.8 | 4.6 | 5.2 | 3.9 | |
| (95% CI) | (4.5-4.9) | (4.6-5.1) | (4.3-4.9) | (4.4-6.0) | (3.5-4.3) | |
| Mean ± SE | 4.7 ± 0.1 | 4.8 ± 0.1 | 4.6 ± 0.2 | 5.2 ± 0.4 | 3.9 ± 0.2 | |
| PTGS2 | | | | | | 0.001 |
| Mean | 6.1 | 6.2 | 5.7 | 7.6 | 5.5 | |
| (95% CI) | (5.8-6.3) | (5.9-6.5) | (5.3-6.1) | (6.2-9.0) | (4.8-6.3) | |
| Mean ± SE | 6.1 ± 0.1 | 6.2 ± 0.2 | 5.7 ± 0.2 | 7.6 ± 0.7 | 5.5 ± 0.4 | |
| RGS1 | | | | | | <.001 |
| Mean | 5.9 | 6.2 | 5.5 | 7.2 | 5.1 | |
| (95% CI) | (5.7-6.2) | (5.9-6.5) | (5.1-5.9) | (6.2-8.2) | (4.4-5.8) | |
| Mean ± SE | 5.9 ± 0.1 | 6.2 ± 0.1 | 5.5 ± 0.2 | 7.2 ± 0.5 | 5.1 ± 0.4 | |

Transcriptome Changes of Immune Genes and Immune Pathway Genes in SLNs Associated with Recurrence in yr⁶⁰⁻ and yr⁶⁰⁺ Melanoma Patients by NanoString Analysis

In the NanoString dataset, we first analyzed the differentially expressed immune genes between recur^{yes} and recur^{no} groups in younger melanoma patients (yr⁶⁰⁻). The results showed that there were 20 differentially expressed immune genes (p<0.05) in this comparison (Supplementary Table 3). Selected differentially expressed immune genes between the recur^{yes} and recur^{no} patients with p<0.05 and absolute fold change >0.5 in the yr⁶⁰⁻ group were listed in Table 6. In yr⁶⁰⁻ patients with positive SLNs, highly expressed C6, interleukin 23 receptor (IL23R), B melanoma antigen (BAGE), chemokine [C-C motif] ligand 16 (CCL16), and lower expression of S100 calcium binding protein B (S100B) were associated with recur^{yes} patients.

In older patients, there were 20 differentially expressed genes between the recur^{yes} and recur^{no} group (p<0.05) (Supplementary Table 4). Table 7 lists the selected differentially expressed immune genes by recurrence status in the yr⁶⁰⁺ melanoma patients with p<0.05 and absolute fold change >0.5. In yr⁶⁰⁺ patients with positive SLNs, highly expressed FOS and CCL18 were associated with recur^{yes}.

When comparing the difference in the DEGs by recurrence status in both age groups, we found that MAPK11 was expressed more highly in the younger melanoma patients in the recur^{yes} versus the recur^{no} group (FC=2.84) (Table 6). A similar family member, MAP2K4, had marginal expression in older patients in the recur^{yes} versus the recur^{no} group (FC=0.25) (Supplementary Table 4). CCL16 had a higher expression in the younger patient cohort in the recur^{yes} versus the recur^{no} group (FC=3.46) (Table 6). Another family member, CCL18, also had a higher expression in older patients with recurrence (FC=1.8) (Table 7). C6 was more highly expressed in younger melanoma patients with recurrence (FC=4.28) (Table 6), while C3 had marginal expression in older patients with recurrence (FC=0.83) (Table 7).

In terms of immune pathway genes, there were 18 differentially expressed genes with p<0.05 and absolute fold change >0.5 in the younger patients when comparing recur^{yes} versus recur^{no} (Table 8). A complete list of the DEGs with p<0.05 is presented in Supplementary Table 5. In the group of older patients, there were 13 differentially expressed immune pathway genes with p<0.05 and absolute fold change >0.5 by recurrence status (Table 9). All the DEGs with p<0.05 in the older group are listed in Supplementary Table 6. IRAK3 (interleukin-1 receptor-associated kinase 3) was the major immune pathway gene found in younger patients with recurrence (Table 8), while Wnt10b was the major pathway found in older patients with recurrence (Table 9). There were no overlapped immune pathway genes in either age group by recurrence status. These results suggested that, even though some immune genes have similar changes in older and younger patients, different pathways may be involved in recurrence in different age groups.

SUPPLEMENTARY TABLE 3
Differentially expressed immune genes in younger patients between the
recur^{yes} and the recur^{no} group by NanoString analysis (p < 0.05).

| | | | Fold |
|---|---|---|---|
| Gene symbol | Gene Name | P value | change |
| C6 | complement component 6 | 0.00745 | 4.28 |
| IL23R | interleukin 23 receptor | 0.00545 | 3.64 |
| BAGE | B melanoma antigen | 0.0136 | 3.58 |
| CCL16 | chemokine (C-C motif) ligand 16 | 0.0168 | 3.46 |
| SPINK5 | serine peptidase inhibitor, Kazal type 5 | 0.0161 | 2.96 |
| MAPK11 | mitogen-activated protein kinase 11 | 0.00968 | 2.84 |
| MST1R | macrophage stimulating 1 receptor (c-met-related | 0.00947 | 2.52 |
| | tyrosine kinase) | | |
| F2RL1 | coagulation factor II (thrombin) receptor-like 1 | 0.00399 | 1.97 |
| DOCK9 | dedicator of cytokinesis 9 | 0.00847 | 1.61 |
| IGF1R | insulin-like growth factor 1 receptor | 0.0141 | 0.971 |
| TBK1 | TANK-binding kinase 1 | 0.0116 | 0.616 |
| MAP2K2 | mitogen-activated protein kinase kinase 2 | 0.00105 | −0.306 |
| HLA-A | major histocompatibility complex, class I, A | 0.00051 | −0.386 |
| CCL4 | chemokine (C-C motif) ligand 4 | 0.0165 | −0.485 |
| ICAM1 | intercellular adhesion molecule 1 | 0.0143 | −0.587 |
| C1QBP | complement component 1, q subcomponent binding | 0.0131 | −0.9 |
| | protein | | |
| PSMB8 | proteasome (prosome, macropain) subunit, beta type, | 0.0121 | −0.939 |
| | 8 (large multifunctional peptidase 7) | | |
| MIF | macrophage migration inhibitory factor | 0.016 | −1.09 |
| | (glycosylation-inhibiting factor) | | |
| HLA-G | major histocompatibility complex, class I, G | 0.00237 | −1.18 |
| S100B | S100 calcium binding protein B | 0.00859 | −5.64 |

TABLE 6
Selected differentially expressed immune genes between
recur^{yes} and recur^{no} group in younger patients (yr⁶⁰⁻)
by NanoString analysis (p < 0.05, absolute fold change >0.5).

| | | | Fold |
|---|---|---|---|
| Gene symbol | Gene Name | P value | change |
| C6 | complement component 6 | 0.00745 | 4.28 |
| IL23R | interleukin 23 receptor | 0.00545 | 3.64 |
| BAGE | B melanoma antigen | 0.0136 | 3.58 |
| CCL16 | chemokine (C-C motif) ligand 16 | 0.0168 | 3.46 |
| SPINK5 | serine peptidase inhibitor, Kazal type 5 | 0.0161 | 2.96 |
| MAPK11 | mitogen-activated protein kinase 11 | 0.00968 | 2.84 |
| MST1R | macrophage stimulating 1 receptor (c-met-related | 0.00947 | 2.52 |
| | tyrosine kinase) | | |
| F2RL1 | coagulation factor II (thrombin) receptor-like 1 | 0.00399 | 1.97 |
| DOCK9 | dedicator of cytokinesis 9 | 0.00847 | 1.61 |
| IGF1R | insulin-like growth factor 1 receptor | 0.0141 | 0.971 |
| TBK1 | TANK-binding kinase 1 | 0.0116 | 0.616 |
| ICAM1 | intercellular adhesion molecule 1 | 0.0143 | −0.587 |
| C1QBP | complement component 1, q subcomponent binding | 0.0131 | −0.9 |
| | protein | | |
| PSMB8 | proteasome (prosome, macropain) subunit, beta | 0.0121 | −0.939 |
| | type, 8 (large multifunctional peptidase 7) | | |
| MIF | macrophage migration inhibitory factor | 0.016 | −1.09 |
| | (glycosylation-inhibiting factor) | | |
| HLA-G | major histocompatibility complex, class I, G | 0.00237 | −1.18 |
| S100B | S100 calcium binding protein B | 0.00859 | −5.64 |

SUPPLEMENTARY TABLE 4
Differentially expressed immune genes in older patients between the
recur^{yes} and the recur^{no} group by NanoString analysis (p < 0.05).

| | | | Fold |
|---|---|---|---|
| Gene symbol | Gene Name | P value | change |
| FOS | FBJ murine osteosarcoma viral oncogene homolog | 0.0221 | 1.9 |
| CCL18 | chemokine (C-C motif) ligand 18 (pulmonary and | 0.00867 | 1.8 |
| | activation-regulated) | | |
| CXCR4 | chemokine (C-X-C motif) receptor 4 | 0.0238 | 1.07 |
| C3 | complement component 3 | 0.00481 | 0.832 |
| TLR10 | toll-like receptor 10 | 0.0189 | 0.787 |
| NOD1 | nucleotide-binding oligomerization domain | 0.00347 | 0.768 |
| | containing 1 | | |
| PLAU | plasminogen activator, urokinase | 0.00371 | 0.741 |
| CYBB | cytochrome b-245, beta polypeptide | 0.00314 | 0.732 |
| TLR6 | toll-like receptor 6 | 0.013 | 0.626 |
| HLA-DMA | major histocompatibility complex, class II, | 0.0192 | 0.606 |
| | DM alpha | | |
| TNFRSF13B | tumor necrosis factor receptor superfamily, | 0.0191 | 0.555 |
| | member 13B | | |
| CD84 | CD84 molecule | 0.014 | 0.504 |
| ATG7 | autophagy related 7 | 0.00748 | 0.486 |
| HLA-DMB | major histocompatibility complex, class II, | 0.0199 | 0.313 |
| | DM beta | | |
| MAP2K4 | mitogen-activated protein kinase kinase 4 | 0.00196 | 0.248 |
| INPP5D | inositol polyphosphate-5-phosphatase, 145 kDa | 0.0117 | 0.216 |
| ELK1 | ELK1, member of ETS oncogene family | 0.00172 | −0.491 |
| RELA | v-rel reticuloendotheliosis viral oncogene | 0.0225 | −0.52 |
| | homolog A (avian) | | |
| IFITM1 | interferon induced transmembrane protein 1 | 0.0176 | −0.675 |
| NCAM1 | neural cell adhesion molecule 1 | 0.00896 | −0.984 |

TABLE 7
Selected differentially expressed immune genes between the recur^{yes} and recur^{no} group
in yr⁶⁰⁺ patients by NanoString analysis (p < 0.05, absolute fold change >0.5).

| | | | Fold |
|---|---|---|---|
| Gene symbol | Gene Name | P value | change |
| FOS | FBJ murine osteosarcoma viral oncogene homolog | 0.0221 | 1.9 |
| CCL18 | chemokine (C-C motif) ligand 18 (pulmonary and | 0.00867 | 1.8 |
| | activation-regulated) | | |
| CXCR4 | chemokine (C-X-C motif) receptor 4 | 0.0238 | 1.07 |
| C3 | complement component 3 | 0.00481 | 0.832 |
| TLR10 | toll-like receptor 10 | 0.0189 | 0.787 |
| NOD1 | nucleotide-binding oligomerization domain | 0.00347 | 0.768 |
| | containing 1 | | |
| PLAU | plasminogen activator, urokinase | 0.00371 | 0.741 |
| CYBB | cytochrome b-245, beta polypeptide | 0.00314 | 0.732 |
| TLR6 | toll-like receptor 6 | 0.013 | 0.626 |
| HLA-DMA | major histocompatibility complex, class II, DM alpha | 0.0192 | 0.606 |
| TNFRSF13B | tumor necrosis factor receptor superfamily, member | 0.0191 | 0.555 |
| | member 13B | | |
| CD84 | CD84 molecule | 0.014 | 0.504 |
| RELA | v-rel reticuloendotheliosis viral oncogene | 0.0225 | −0.52 |
| | homolog A (avian) | | |
| IFITM1 | interferon induced transmembrane protein 1 | 0.0176 | −0.675 |
| NCAM1 | neural cell adhesion molecule 1 | 0.00896 | −0.984 |

TABLE 8
Differentially expressed immune pathway genes in younger patients
(yr⁶⁰⁻) between the recur^{yes} and recur^{no} group by NanoString
analysis (p < 0.05, absolute fold change >0.5).

| | | | Fold |
|---|---|---|---|
| Gene symbol | Gene Name | P value | change |
| IRAK3 | interleukin-1 receptor-associated kinase 3 | 0.00552 | 2.15 |
| NKD1 | naked cuticle homolog 1 (Drosophila) | 0.00565 | 2.13 |
| ACVRIC | activin A receptor, type IC | 0.0111 | 1.9 |
| SOS1 | son of sevenless homolog 1 (Drosophila) | 0.00681 | 1.42 |
| EPOR | erythropoietin receptor | 0.00773 | 1.32 |
| ACVR2A | activin A receptor, type IIA | 0.012 | 1.12 |
| RAD50 | RAD50 homolog (S. cerevisiae) | 0.0101 | 0.83 |
| SMAD2 | SMAD family member 2 | 0.00233 | 0.799 |
| DNMT3A | DNA (cytosine-5-)-methyltransferase 3 alpha | 0.0113 | 0.745 |
| RPS6KA5 | ribosomal protein S6 kinase, 90 kDa, polypeptide 5 | 0.00593 | 0.596 |
| FANCL | Fanconi anemia, complementation group L | 0.00732 | −0.554 |
| PPP2R1A | protein phosphatase 2, regulatory subunit A, alpha | 0.00791 | −0.679 |
| RB1 | retinoblastoma 1 | 0.0108 | −0.84 |
| UBB | ubiquitin B | 0.0109 | −0.849 |
| CDK4 | cyclin-dependent kinase 4 | 0.00456 | −1.22 |
| CASP9 | caspase 9, apoptosis-related cysteine peptidase | 0.00969 | −1.22 |
| HSP90B1 | heat shock protein 90 kDa beta (Grp94), member 1 | 0.0106 | −1.25 |
| PCNA | proliferating cell nuclear antigen | 0.00314 | −1.45 |

SUPPLEMENTARY TABLE 5
Differentially expressed immune pathway genes in younger patients between
the recur^{yes} and the recur^{no} group by NanoString analysis (p < 0.05).

| | | | Fold |
|---|---|---|---|
| Gene symbol | Gene Name | P value | change |
| IRAK3 | interleukin-1 receptor-associated kinase 3 | 0.00552 | 2.15 |
| NKD1 | naked cuticle homolog 1 (Drosophila) | 0.00565 | 2.13 |
| ACVRIC | activin A receptor, type IC | 0.0111 | 1.9 |
| SOS1 | son of sevenless homolog 1 (Drosophila) | 0.00681 | 1.42 |
| EPOR | erythropoietin receptor | 0.00773 | 1.32 |
| ACVR2A | activin A receptor, type IIA | 0.012 | 1.12 |
| RAD50 | RAD50 homolog (S. cerevisiae) | 0.0101 | 0.83 |
| SMAD2 | SMAD family member 2 | 0.00233 | 0.799 |
| DNMT3A | DNA (cytosine-5-)-methyltransferase 3 alpha | 0.0113 | 0.745 |
| RPS6KA5 | ribosomal protein S6 kinase, 90 kDa, polypeptide 5 | 0.00593 | 0.596 |
| MAP2K2 | mitogen-activated protein kinase kinase 2 | 0.00454 | −0.363 |
| PIK3R3 | phosphoinositide-3-kinase, regulatory subunit 3 | 0.00981 | −0.439 |
| | (gamma) | | |
| FANCL | Fanconi anemia, complementation group L | 0.00732 | 0.554 |
| PPP2R1A | protein phosphatase 2, regulatory subunit A, alpha | 0.00791 | −0.679 |
| RB1 | retinoblastoma 1 | 0.0108 | −0.84 |
| UBB | ubiquitin B | 0.0109 | −0.849 |
| CDK4 | cyclin-dependent kinase 4 | 0.00456 | −1.22 |
| CASP9 | caspase 9, apoptosis-related cysteine peptidase | 0.00969 | −1.22 |
| HSP90B1 | heat shock protein 90 kDa beta (Grp94), member 1 | 0.0106 | −1.25 |
| PCNA | proliferating cell nuclear antigen | 0.00314 | −1.45 |

TABLE 9
Differentially expressed immune pathway genes in older patients
(yr⁶⁰⁺) between the recur^{yes} and recur^{no} group by NanoString
analysis (p < 0.05, absolute fold change >0.5).

| | | | Fold |
|---|---|---|---|
| Gene symbol | Gene Name | P value | change |
| WNT10B | wingless-type MMTV integration site family, | 0.027 | 2.27 |
| | member 10B | | |
| HSPA1A | heat shock 70 kDa protein 1A | 0.0283 | 2.04 |
| FOS | FBJ murine osteosarcoma viral oncogene homolog | 0.0219 | 1.96 |
| DKK2 | dickkopf WNT signaling pathway inhibitor 2 | 0.0247 | 1.7 |
| IL6 | interleukin 6 (interferon, beta 2) | 0.00111 | 1.36 |
| TGFB3 | transforming growth factor, beta 3 | 0.0379 | 1.19 |
| HHEX | hematopoietically expressed homeobox | 0.0263 | 1.06 |
| DLL4 | delta-like 4 (Drosophila) | 0.00752 | 0.883 |
| XRCC4 | X-ray repair complementing defective repair in | 0.0354 | 0.787 |
| | Chinese hamster cells 4 | | |
| NR4A1 | nuclear receptor subfamily 4, group A, member 1 | 0.0232 | 0.766 |
| ALKBH2 | alkB, alkylation repair homolog 2 (E. coli) | 0.0384 | 0.752 |
| PLAU | plasminogen activator, urokinase | 0.00195 | 0.659 |
| BID | BH3 interacting domain death agonist | 0.0369 | 0.564 |

SUPPLEMENTARY TABLE 6
Differentially expressed immune pathway genes in older patients between
the recur^{yes} and the recur^{no} group by NanoString analysis (p < 0.05).

| | | | Fold |
|---|---|---|---|
| Gene symbol | Gene Name | P value | change |
| WNT10B | wingless-type MMTV integration site family, | 0.027 | 2.27 |
| | member 10B | | |
| HSPA1A | heat shock 70 kDa protein 1A | 0.0283 | 2.04 |
| FOS | FBJ murine osteosarcoma viral oncogene homolog | 0.0219 | 1.96 |
| DKK2 | dickkopf WNT signaling pathway inhibitor 2 | 0.0247 | 1.7 |
| IL6 | interleukin 6 (interferon, beta 2) | 0.00111 | 1.36 |
| TGFB3 | transforming growth factor, beta 3 | 0.0379 | 1.19 |
| HHEX | hematopoietically expressed homeobox | 0.0263 | 1.06 |
| DLL4 | delta-like 4 (Drosophila) | 0.00752 | 0.883 |
| XRCC4 | X-ray repair complementing defective repair | 0.0354 | 0.787 |
| | in Chinese hamster cells 4 | | |
| NR4A1 | nuclear receptor subfamily 4, group A, member 1 | 0.0232 | 0.766 |
| ALKBH2 | alkB, alkylation repair homolog 2 (E. coli) | 0.0384 | 0.752 |
| PLAU | plasminogen activator, urokinase | 0.00195 | 0.659 |
| BID | BH3 interacting domain death agonist | 0.0369 | 0.564 |
| ERCC6 | excision repair cross-complementing rodent | 0.0236 | 0.479 |
| | repair deficiency, complementation group 6 | | |
| LAMA5 | laminin, alpha 5 | 0.00181 | 0.467 |
| NFKBIZ | nuclear factor of kappa light polypeptide | 0.00751 | 0.418 |
| | gene enhancer in B-cells inhibitor, zeta | | |
| RUNX1 | runt-related transcription factor 1 | 0.0281 | 0.417 |
| TFDP1 | transcription factor Dp-1 | 0.0364 | 0.36 |
| STK11 | serine/threonine kinase 11 | 0.0186 | 0.312 |
| BCOR | BCL6 corepressor | 0.0379 | −0.377 |

Verification of the DEGs in the Third Independent Dataset by Quantitative Reverse Transcriptase Polymerase Chain Reaction (qRT-PCR)

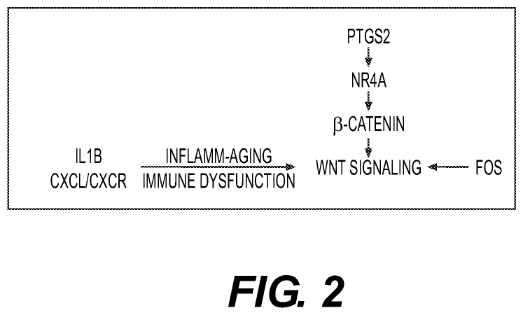

After using microarray and NanoString technologies to identify the DEGs in the yr⁶⁰⁻ and yr⁶⁰⁺ patients as well as the different outcome-associated age groups (recur^{yes} versus recur^{no}), we used qRT-PCR in another independent dataset to confirm the findings above. We selected genes that were differentially expressed in both microarray and NanoString analysis or had higher fold changes in either of the analysis. The results showed that FOS, NR4A2, PTGS2, LINC00518, IL1B, and Wnt10b were all highly expressed in older patients with recurrence (Table 10). These genes converged at the Wnt10b pathway (FIG. 2).

TABLE 10
qRT-PCR Validation of the DEGs in the third
independent dataset (recur^{yes} versus recur^{no})

Fold Change

| Gene name | Age <60 | Age ≥60 |
|---|---|---|
| FOS | +1.2 | +12.6 |
| NR4A2 | +1.4 | +4.2 |
| PTGS2 | +1.8 | +3.5 |
| LINC00518 | +3.2 | +9.0 |
| IL1B | −1.2 | +1.1 |
| Wnt10b | +1.6 | +2.9 |
| CCL18 | −1.7 | +1.3 |
| HSPA1A | −1.2 | +1.3 |
| NRCAM | +1.14 | +2.0 |
| CXCL5 | +1.4 | +1.6 |

### Discussion

In this study, we used three independent datasets and two different technologies, microarray and NanoString, to identify the DEGs in SLNs that are associated with recurrence by age group. NanoString used a novel method of direct mRNA barcoding and digital detection of target molecules through the use of color-coded probe pairs. This new technology does not need reverse transcription and the downstream PCR amplification to assess the gene expression level. We selected an immune panel and an immune pathway panel for NanoString analysis to focus on immune-related gene changes in SLNs. The results showed that there was some overlap of DEGs (NR4A and FOS) that have been detected by both technologies. Those genes have been confirmed by PCR in an independent dataset. Some genes (PTGS2, IL1B, LINC00518, and Wnt10b) that have higher fold changes detected by either of the two technologies were also confirmed by PCR in an independent dataset. The two technologies complement each other. In combination with the three independent datasets used in this study, these data provide a higher standard of research integrity.

Our results showed that Wnt signaling and related genes in SLNs have changes that correlate with recurrence in older melanoma patients with SLN metastasis. We found that SFRP2 and SFRP4 has high fold change genes in older melanoma patients compared with their younger counterparts.

Currently, no reports have shown how the PTGS2-NR4A-Wnt network is associated with age-related immunity in melanoma. In our study, we found that Wnt10b was upregulated in older melanoma patients who experience recurrence. The upstream genes of PTGS2 and NR4A were also upregulated.

Our results showed that FOS was the gene with a high fold change in recur^{yes} versus recur^{no} in older melanoma patients. Upregulated FOS might occur in conjunction with activated Wnt pathway to promote melanoma progression in older patients.

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner Indeed, various modifications of the invention in addition to those described herein will become apparent to those skilled in the art from the foregoing description and the accompanying figures. Such modifications are intended to fall within the scope of the appended claims.

What is claimed is:

1. A method for treating melanoma in a subject, the method comprising:
quantifying an RNA expression level for at least one biomarker in a sample from a sentinel lymph node of the subject, where the at least one biomarker comprises FOS, NR4A, ITGB1, IRAK3, Wnt10b, or a combination thereof, and
administering to the subject immunotherapy, interferon, a BRAF inhibitor, a checkpoint inhibitor, a Wnt10b inhibitor, an IRAK3 inhibitor, or a combination thereof.

2. The method of claim 1, wherein the at least one biomarker comprises NR4A1, NR4A2, NR4A3, or a combination thereof.

3. The method of claim 1 or claim 2, wherein the at least one biomarker comprises NR4A2, NR4A3, or both.

4. The method of any of the preceding claims, wherein the at least one biomarker comprises NR4A, FOS, Wn10b, or a combination thereof.

5. The method of any of the preceding claims, wherein the at least one biomarker comprises NR4A.

6. The method of any of the preceding claims, wherein the at least one biomarker comprises IRAK3.

7. The method of any of the preceding claims, wherein the at least one biomarker further comprises SFRP2, SFRP4, PTGS2, LINC00518, IL1B, or a combination thereof.

8. The method of any of the preceding claims, wherein the at least one biomarker further comprises a biomarker listed in Table 2, Table 3, Table 4, Table 5, Table 6, Table 7, Table 8, Table 9, Table 10, Supp. Table 2, Supp. Table 3, Supp. Table 4, Supp. Table 5, Supp. Table 6, or a combination thereof.

9. The method of any of the preceding claims, wherein the at least one biomarker further comprises ACVR1C, ACVR2A, ALCAM, ALKBH2, ATG7, ATP2B2, BACH2, BAGE, BCOR, BID, C1QBP, C3, C6, CASP9, CCL16, CCL18, CCL4, CD244, CD84, CD8A, CDK4, CDK6, CDKN1A, CLEC4C, CLEC7A, COL28A1, COMP, CTNNB1, CXL/CXCR, CXCL3, CXCL5, CXCR4, CYBB, DKK2, DLK1, DLL4, DNMT3A, DOCK9, DUSP1, ELK1, EPOR, ERCC2, ERCC6, ERGIC3, F2RL1, FANCB, FANCL, FOS, FOSB, FUT8, GADD45A, GRB14, GRIK2, HHEX, HLA-A, HLA-DMA, HLA-DMB, HLA-G, HSP90B1, HSPA1A, ICAM1, IDO1, IFIH1, IFITM1, IGF1R, IL1B, IL23R, IL6, INHBA, INPP5D, IRAK3, ITGB1, ITGBL1, JAM3, KLF4, KLRC4-KLRK1///KLRK1, LAMA5, LIF, LINC00354, LINC00518, LIX1, LOC100507516, LOC101928963, LOC105373225, MAGEA3, MAP2K2, MAP2K4, MAPK11, MAVS, MIF, MKX, MLANA, MME, MS4A6A, MST1R, MUC15, MX1, NCAM1, NFKBIZ, NKD1, NOD1, NOG, NR4A, NR4A1, NR4A2, NR4A3, NRCAM, PBRM1, PCNA, PIK3CB, PIK3R3, PLAU, PLD1, PPP2R1A, PRKAR2A, PRUNE2, PSMB8, PTGS2, RAD50, RB1, RELA, RGS1, RNF152, RPS6KA5, RUNX1, S100B, SATB1, SFRP2, SFRP4, SLC13A5, SMAD2, SOS1, SPINK5, STK11, TBK1, TFDP1, TFPI2, TGFB3, TLR10, TLR6, TNC, TNFRSF10C, TNFRSF13B, UBB, WNT10B, WWC1, XRCC4, or a combination thereof.

10. The method of any of the preceding claims, wherein the quantifying is carried out using polymerase chain reaction, real-time polymerase chain reaction, reverse transcriptase polymerase chain reaction, real-time quantitative RT-PCR, microarray, NanoString, or a combination thereof.

11. The method of any of the preceding claims, wherein the quantifying is carried out using polymerase chain reaction, real-time polymerase chain reaction, reverse transcriptase polymerase chain reaction, real-time quantitative RT-PCR, or a combination thereof.

12. The method of any of the preceding claims, wherein the subject is no more than about 30 years old, no more than about 40 years old, no more than about 50 years old, no more than about 60 years old, or no more than about 70 years old.

13. The method of any of the preceding claims, wherein the subject is at least about 40 years old, at least about 50 years old, at least about 60 years old, at least about 70 years old, or at least about 80 years old.

14. The method of any of the preceding claims, wherein the subject has a positive sentinel lymph node status.

15. The method of any of the preceding claims, wherein the quantifying is relative to a control sample where the melanoma was without recurrence for at least about 5.0 years.

16. The method of any of the preceding claims, wherein the fold change in the RNA expression level, relative to a control sample where the melanoma was without recurrence for at least about 5.0 years, in one or more of the at least one biomarker is at least about 1.0, at least about 1.5, at least about 2.0, at least about 2.5, at least about 3.0, at least about 3.5, at least about 4.0, or at least about 4.5.

17. The method of any of the preceding claims, wherein the method further comprises assessing a clinicopathologic feature of the subject from which the sample was obtained.

18. The method of any of the preceding claims, wherein the method further comprises assessing a clinicopathologic feature of the subject from which the sample was obtained and the clinicopathologic feature is age, gender, anatomic location, Breslow thickness, ulceration, sentinel lymph node status, or a combination thereof.

19. The method of any of the preceding claims, wherein the method further comprises assessing a clinicopathologic feature of the subject from which the sample was obtained and the clinicopathologic feature is metastasis, age, lesion site, tumor burden, number of positive nodes, ulceration, tumor thickness, or a combination thereof.

20. The method of any of the preceding claims, wherein the subject has stage III 10 melanoma.

21. The method of any of the preceding claims, wherein the administering comprises administering interferon-gamma, interferon alfa-2b, or both.

22. The method of any of the preceding claims, wherein the administering comprises administering a BRAF inhibitor, vemurafenib, dabrafenib, trametinib, encorafenib, or a combination thereof.

23. The method of any of the preceding claims, wherein the administering comprises administering a checkpoint inhibitor, a PD-1 inhibitor, nivolumab, pembrolizumab, cemiplimab, a PD-L1 inhibitor, atezolizumab, avelumab, durvalumab, a cytotoxic T-lymphocyte antigen 4 inhibitor, ipilimumab, or a combination thereof.

24. The method of any of the preceding claims, wherein the administering comprises administering a Wnt10b inhibitor, DKN-01, CGX1321, ETC 1922159, or a combination thereof.

25. The method of any of the preceding claims, wherein (a) the administering comprises administering a Wnt10b inhibitor, DKN-01, CGX1321, ETC 1922159 or a combination thereof and (b) the subject is at least about 60 years old.

26. The method of any of the preceding claims, wherein the administering comprises administering an IRAK3 inhibitor, pacritinib, thymoquinone, or a combination thereof.

27. The method of any of the preceding claims, wherein (a) the administering comprises administering an IRAK3 inhibitor, pacritinib, thymoquinone, or a combination thereof and (b) the subject is no more than about 60 years old.

28. The method any of the preceding claims, wherein the administering only occurs if the fold change in the RNA expression level, relative to a control sample where the melanoma was without recurrence for at least about 5.0 years, in one or more of the at least one biomarker is at least about 1.0, at least about 1.5, at least about 2.0, at least about 2.5, at least about 3.0, at least about 3.5, at least about 4.0, or at least about 4.5.

29. The method any of the preceding claims, wherein the administering comprises administering a Wnt10b inhibitor, DKN-01, CGX1321, ETC 1922159, or a combination thereof, and the administering only occurs if (a) the fold change in the RNA expression level, relative to a control sample where the melanoma was without recurrence for at least about 5.0 years, in one or more of the at least one biomarker is at least about 1.0, at least about 1.5, at least about 2.0, at least about 2.5, at least about 3.0, at least about 3.5, at least about 4.0, or at least about 4.5 and (b) the subject is at least about 60 years old.

30. The method any of the preceding claims, wherein the administering comprises administering of IRAK3 inhibitor, pacritinib, thymoquinone, or a combination thereof, and the administering only occurs if (a) the fold change in the RNA expression level, relative to a control sample where the melanoma was without recurrence for at least about 5.0 years, in one or more of the at least one biomarker is at least about 1.0, at least about 1.5, at least about 2.0, at least about 2.5, at least about 3.0, at least about 3.5, at least about 4.0, or at least about 4.5 and (b) the subject is no more than about 60 years old.

31. The method any of the preceding claims, wherein the treating further comprises surgery, chemotherapy, radiation therapy, targeted therapy, vaccine therapy, or a combination thereof.

32. The method any of the preceding claims, wherein the subject is a mammal, a primate, or a human.

33. The method any of the preceding claims, wherein the subject is a human.

34. A method for treating melanoma in a human, the method comprising:
quantifying an RNA expression level for at least one biomarker in a sample from a sentinel lymph node of the human, where the at least one biomarker comprises FOS, NR4A, Wnt10b, or a combination thereof, and
administering to the human a Wnt10b inhibitor, DKN-01, CGX1321, ETC 1922159, or a combination thereof, and optionally one or more of immunotherapy, interferon, a BRAF inhibitor, a checkpoint inhibitor, or an IRAK3 inhibitor;
wherein
the human is at least about 60 years old, and
the administering only occurs if the fold change in the RNA expression level, relative to a control sample where the melanoma was without recurrence for at least about 5.0 years, in one or more of the at least one biomarker is at least about 1.5.

35. A method for treating melanoma in a human, the method comprising:
quantifying an RNA expression level for at least one biomarker in a sample from a sentinel lymph node of the human, where the at least one biomarker comprises IRAK3, and
administering to the human an IRAK3 inhibitor, pacritinib, thymoquinone, or a combination thereof, and optionally one or more of immunotherapy, interferon, a BRAF inhibitor, a checkpoint inhibitor, or a Wnt10b inhibitor;
wherein
the human is no more than about 60 years old, and
the administering only occurs if the fold change in the RNA expression level, relative to a control sample where the melanoma was without recurrence for at least about 5.0 years, in one or more of the at least one biomarker is at least about 1.5.
